(19) United States
(12) Patent Application Publication
(10) Pub. No.: US 2023/0136558 A1
(43) Pub. Date: May 4, 2023

(54) SYSTEMS AND METHODS FOR MACHINE VISION ANALYSIS

(71) Applicant: Avail Medsystems, Inc., Santa Clara, CA (US)

(72) Inventors: Daniel HAWKINS, Palo Alto, CA (US); Ravi KALLURI, San Jose, CA (US); Arun KRISHNA, Pleasanton, CA (US); Shivakumar MAHADEVAPPA, Fremont, CA (US)

(21) Appl. No.: 18/046,720

(22) Filed: Oct. 14, 2022

Related U.S. Application Data
(63) Continuation of application No. PCT/US21/36389, filed on Jun. 8, 2021.
(60) Provisional application No. 63/036,769, filed on Jun. 9, 2020.

(51) Int. Cl. G16H 40/20; G06T 7/00; A61B 34/00

(52) U.S. Cl. CPC G16H 40/20; G06T 7/0012; A61B 34/25; G06T 2207/10016; G06T 2207/30004; A61B 2034/252

(57) ABSTRACT

Systems and methods for machine vision/audio use and analysis are provided. Systems and methods are provided for identifying medical conditions based on data collected during a procedure. Systems and methods are provided for providing recommendations relating to procedures based on data collected during a procedure. Furthermore, recommendations may be provided relating to products and potential improvements based on data collected during a procedure.

### CROSS-REFERENCE

This application is a continuation of International Patent Application PCT/US21/36389, filed on Jun. 8, 2021, which claims priority to U.S. Provisional Patent Application No. 63/036,769, filed on Jun. 9, 2020, each of which is incorporated herein by reference in its entirety for all purposes.

### BACKGROUND OF THE INVENTION

Medical procedures may be performed in response to a known condition of a patient. During the procedure, the known condition may be treated but traditional systems and methods may not collect significant new data during a procedure, or use such data in an effective manner, which may provide less than optimal patient outcomes.

### SUMMARY OF THE INVENTION

A need exists for improved systems and methods for machine vision analysis. A need exists for systems and methods that allow for identifying medical conditions based on data collected during a procedure. A further need exists for providing recommendations relating to procedures based on data collected during a medical procedure. Additionally, a need exists for systems and methods that may provide recommendations relating to medical products based on data collected during a medical procedure.

Aspect of the invention are directed to a method of forecasting usage of one or more medical resources, said method comprising: collecting, with aid of one or more video systems, images of a patient during a procedure at a health care location; analyzing, with aid of one or more processors the images collected with aid of the one or more video systems of the patient during the procedure at the health care location; recognizing, with aid of the one or more processors, a medical condition of the patient based on the analyzed images collected by the video systems; and alerting medical personnel to the recognized medical condition.

In some embodiments, the medical condition is previously undetected for the patient. Optionally, the medical condition is recognized during the procedure. The method may further comprise generating and recommending, with aid of the one or more processors, next steps for the procedure, based on the images collected or audio data collected during the procedure. The method may further comprise detecting and identifying, with aid of the one or more processors, one or more medical products during the procedure based on the images collected or audio data collected during the procedure. The method may further comprise recommending, with aid of the one or more processors, one or more medical products to use during the procedure.

Aspects of the invention may be further directed to a method of formulating product recommendations, said method comprising: collecting, with aid of one or more video or audio systems, images or audio of a patient during a procedure at a health care location; analyzing, with aid of one or more processors the images or audio collected with aid of the one or more video or audio systems of the patient during the procedure at the health care location; and creating, with aid of one or more processors, new medical products or suggesting modifications to existing medical products based on the analysis of the images or audio collected during the procedure. In some embodiments, the method may further comprise providing smart accounting of medical products during the procedure.

In another aspect, the present disclosure provides a method for forecasting usage of one or more medical resources, comprising: collecting, with aid of one or more video systems, images or videos of a patient during a procedure at a health care location; analyzing, with aid of one or more processors the images or videos collected with aid of the one or more video systems of the patient during the procedure at the health care location; recognizing, with aid of the one or more processors, a medical condition of the patient based on the analyzed images or videos collected by the video systems; and alerting medical personnel to the recognized medical condition. In some embodiments, the medical condition is previously unknown or undetected for the patient. In some embodiments, the medical condition is recognized during the procedure.

In some embodiments, the method may further comprise generating and recommending, with aid of the one or more processors, next steps for the procedure, based on the images collected or audio data collected during the procedure. In some embodiments, the method may further comprise detecting and identifying, with aid of the one or more processors, one or more medical products during the procedure based on the images collected or audio data collected during the procedure. In some embodiments, the one or more medical products comprises one or more medical tools or instruments. In some embodiments, the method may further comprise recommending, with aid of the one or more processors, one or more medical products to use during the procedure. In some embodiments, the method may further comprise detecting or tracking, with aid of the one or more processors, a usage or an operation of the one or more medical products during the procedure, based on the images collected or audio data collected during the procedure. In some embodiments, the method may further comprise recommending one or more optimal ways for performing one or more steps of the procedure based on the detection or identification of the one or more medical products. In some embodiments, the method may further comprise recommending one or more optimal ways for performing one or more steps of the procedure based on the recognized medical condition. In some embodiments, the method may further comprise detecting, identifying, or predicting, with aid of the one or more processors, one or more current or future steps of the procedure. In some embodiments, the method may further comprise recommending a specific product, medical operator, or medical technique based on the recognized condition. In some embodiments, the method may further comprise generating or updating one or more recommendations for the procedure based on a change in the recognized condition. In some embodiments, the one or more recommendations comprise a recommendation for a specific product, a particular medical operator, or a certain medical technique. In some embodiments, the method may further comprise generating one or more recommendations for the procedure based on patient information, wherein the patient information comprises medical records, medical history, or medical information provided by or obtained from the patient. In some embodiments, the method may further comprise generating one or more recommendations for the procedure based on data from auxiliary sources, wherein the auxiliary sources comprise endoscopes, laparoscopes, electrocardiogram (ECG) devices, heartbeat monitors, or pulse oximeters. In some embodiments, the method may further comprise generating one or more real-time recommendations for the procedure as the images or videos are being captured or analyzed. In some embodiments, the method may further comprise generating one or more recommendations for future procedures based on an analysis of a past procedure. In some embodiments, the one or more recommendations may comprise a variation of a medical technique performed in the past procedure. In some embodiments, the method may further comprise ranking one or more variations of the medical technique. In some embodiments, the method may further comprise predicting an outcome for the procedure based on the recognized condition and one or more input parameters. In some embodiments, the one or more input parameters may comprise a medical condition of the patient, one or more tools used to perform the procedure, an identity of medical personnel performing or assisting with the procedure, an identity of remote users, a location of the procedure, or one or more techniques used to perform one or more steps of the procedure. In some embodiments, the method may further comprise recommending one or more products based on a comparison between outcomes or results associated with a plurality of different products.

In another aspect, the present disclosures provides a method for formulating product recommendations, the method comprising: collecting, with aid of one or more video or audio systems, images, video, or audio of a patient during a procedure at a health care location; analyzing, with aid of one or more processors the images, video, or audio collected with aid of the one or more video or audio systems of the patient during the procedure at the health care location; and recommending, with aid of one or more processors, one or more new medical products or modifications to one or more existing medical products based on the analysis of the images, video, or audio collected during the procedure. In some embodiments, the method may further comprise providing smart accounting of medical products during the procedure. In some embodiments, the method may further comprise recommending one or more functionally equivalent products associated with the one or more existing medical products. In some embodiments, the recommendations for the one or more new medical products or the suggestions for modifying the one or more existing medical products are generated based on an analysis of patient outcomes associated with the new or existing medical products. In some embodiments, the recommendations for the one or more new medical products or the suggestions for modifying the one or more existing medical products are generated based on one or more factors associated with product functionality, product usage rate, or cost. In some embodiments, the modifications may comprise an adjustment to dimensions, proportions, shape, materials, instructions for usage, or components. In some embodiments, the method may further comprise updating the recommendations in real time based on an analysis of additional images, video, or audio collected during the procedure. In some embodiments, the method may further comprise predicting a surgical outcome based on the recommendations for the one or more new medical products or the modifications to the one or more existing medical products. In some embodiments, the method may further comprise using a machine learning algorithm to generate the recommendations for the one or more new medical products or the modifications to the one or more existing medical products.

Additional aspects and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of a video capture system, in accordance with embodiments of the invention.

FIG. 2 shows an example of medical products that may be recognized using a video capture system, in accordance with embodiments of the invention.

FIG. 3 shows an example of how video captured may be utilized by an analysis system in order to recommend medical procedure steps, in accordance with embodiments, of the invention.

FIG. 4 shows an example of various types of procedure recommendations that may be formulated by a video analysis system, in accordance with embodiments of the invention.

FIG. 5 shows an example of past procedure analysis and variation recommendations, in accordance with embodiments of the invention.

FIG. 6 shows an example of how various input parameters may affect an updated outcome by a video analysis system, in accordance with embodiments of the invention.

FIG. 7 provides an example of how a video analysis system may automatically detect a medical condition, in accordance with embodiments of the invention.

FIG. 8 provides an example of how various inputs from facilities may be used by the analysis system to provide recommendations to product manufacturers, in accordance with embodiments of the invention.

FIG. 9 shows an example of various recommendations that may be provided to a manufacturer in accordance with embodiments of the invention.

FIG. 10 shows an example of recommendations that may be provided by a medical resource intelligence system for improved performance of a procedure, in accordance with embodiments of the invention.

FIGS. 11A-D show examples of various machine learning techniques that may be utilized, in accordance with embodiments of the invention.

FIG. 11E shows an example of an architecture of the system, in accordance with some embodiment of the present disclosure.

FIG. 12 shows an exemplary computer system, in accordance with embodiments of the invention.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention provides systems and methods for medical resource intelligence. Various aspects of the invention described herein may be applied to any of the particular applications set forth below. The invention may be applied as a part of a health care system or communication system. It shall be understood that different aspects of the invention can be appreciated individually, collectively or in combination with each other.

An analysis system may collect information during one or more medical procedures. In some instances, the collected information may include image data that may be collected with aid of a video capture system. Machine vision/audio systems and methods may be used to identify and/or track usage of products or other resources. Machine vision/audio systems and methods may be coupled with machine learning to recognize products or other resources, and/or activities in relation to a procedure. Any description herein of machine vision systems may apply to audio systems and/or combination machine video/audio systems, and vice versa.

Based on the collected data, an analysis system may make one or more recommendations. Recommendations may be made for imminent or occurring procedures. Recommendations may be made for future procedures. Recommendations may be made relating to past procedures that have been completed. Such recommendations may include different steps that may be performed in relation to the procedure and/or products used. In some instances, recommendations may be made for changes to products themselves, such as adjustments to existing products or designs for new products. The recommendations may be made to yield improved results in relation to procedures.

The collected data may also be useful for detecting medical conditions for patients. For instance, previously unknown conditions may be detected based on data, such as image data, that may be captured prior to, during, or after a procedure. Medical personnel may be alerted to the detected medical condition, which may allow for more rapid and proactive treatment of the patient as needed. Detected medical conditions may also affect recommendations in relation to a past, future, or currently ongoing medical procedure to yield an improved outcome.

The systems and methods provided herein may utilize a video capture system in order to capture images during the surgical procedure.

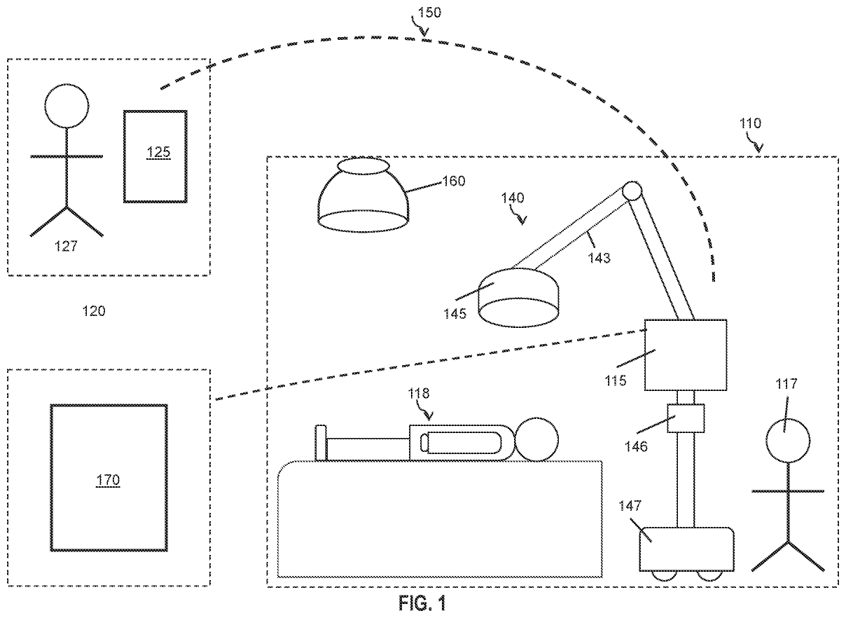

FIG. 1 shows an example of a video capture system utilized within a medical suite, such as an operating room. The video capture system may optionally allow for communications between the medical suite and one or more remote individuals, in accordance with embodiments of the invention. Communication may optionally be provided between a first location 110 and a second location 120.

The first location 110 may be a medical suite, such as an operating room of a health care facility. A medical suite may be within a clinic room or any other portion of a health care facility. A health care facility may be any type of facility or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel/medical practitioner). Any description herein of a health care facility may refer to a hospital or any other type of health care facility, and vice versa.

The first location may be any room or region within a health care facility. For example, the first location may be an operating room, surgical suite, clinic room, triage center, emergency room, or any other location. The first location may be within a region of a room or an entirety of a room. The first location may be any location where an operation may occur, where surgery may take place, where a medical procedure may occur, and/or where a medical product is used. In one example, the first location may be an operating room with a patient 118 that is being operated on, and one or more medical personnel 117, such as a surgeon or surgical assistant that is performing the operation, or aiding in performing the operation. Medical personnel may include any individuals who are performing the medical procedure or aiding in performing the medical procedure. Medical personnel may include individuals who provide support for the medical procedure. For example, the medical personnel may include a surgeon performing a surgery, a nurse, an anesthesiologist, and so forth. Examples of medical personnel may include physicians (e.g., surgeons, anesthesiologists, radiologists, internists, residents, oncologists, hematologists, cardiologists, etc.), nurses (e.g., CNRA, operating room nurse, circulating nurse), physicians' assistants, surgical techs, and so forth. Medical personnel may include individuals who are present for the medical procedure and authorized to be present.

Medical resources may include medical products, medical personnel, locations, instruments, utilities, or any other resource that may be involved for a medical procedure.

Medical products may include devices that are used alone or in combination with other devices for therapeutic or diagnostic purposes. Medical products may be medical devices. Medical products may include any products that are used during an operation to perform the operation or facilitate the performance of the operation. Medical products may include tools, instruments, implants, prostheses, disposables, or any other apparatus, appliance, software, or materials that may be intended by the manufacturer to be used for human beings. Medical products may be used for diagnosis, monitoring, treatment, alleviation, or compensation for an injury or handicap. Medical products may be used for diagnosis, prevention, monitoring, treatment, or alleviation of disease. In some instances, medical products may be used for investigation, replacement, or modification of anatomy or of a physiological process. Some examples of medical products may range from surgical instruments (e.g., handheld or robotic), catheters, endoscopes, stents, pacemakers, artificial joints, spine stabilizers, disposable gloves, gauze, IV fluids, drugs, and so forth.

Medical personnel may be considered as medical resources as well. For example, the number and types of individuals that may be required to be present at a medical procedure may be considered as a medical resource. The identities of the individuals that may be present or providing support remotely may be considered as a medical resource.

A video capture system may have one or more cameras. The video capture system may also comprise a local communication device 115. The local communication device may optionally communicate with a remote communication device 125. The local communication device may be part of a medical console. The local communication device may be integral to or separable from the medical console.

One or more cameras may be integral to the communication device. Alternatively, the one or more cameras may be removable and/or connectable to the communication device. The one or more cameras may face a user when the user looks at a display of the communication device. The one or more cameras may face away from a user when the user looks at a display of the communication device. In some instances, multiple cameras may be provided which may face in different directions. The cameras may be capable of capturing images at a desired resolution. For instance, the cameras may be capable of capturing images at least a 6 mega pixel, 8 mega pixel, 10 mega pixel, 12 mega pixel, 20 mega pixel, 30 megapixels, 40 megapixels, or any number of pixels. The cameras may be capable of capturing SD, HD, Full HD, WUXGA, 2K, UHD, 4K, 8K, or any other level of resolution. A camera on a rep communication device may capture an image of a vendor representative. A camera on a local communication device may capture an image of a medical personnel. A camera on a local communication device may capture an image of a surgical site and/or medical tools, instruments or products.

The communication device may comprise one or more microphones or speakers. A microphone may capture audible noises such as the voice of a user. For instance, the rep communication device microphone may capture the speech of the vendor representative and a local communication device microphone may capture the speech of a medical personnel. One or more speakers may be provided to play sound. For instance, a speaker on a rep communication device may allow a vendor representative to hear sounds captured by a local communication device, and vice versa.

In some embodiments, an audio enhancement module may be provided. The audio enhancement module may be supported by a video capture system. The audio enhancement module may comprise an array of microphones that may be configured to clearly capture voices within a noisy room while minimizing or reducing background noise. The audio enhancement module may be separable or may be integral to the video capture system. The audio enhancement module may be separate or may be integral to a medical console.

A communication device may comprise a display screen. The display screen may be a touchscreen. The display screen may accept inputs by a user's touch, such as finger. The display screen may accept inputs by a stylus or other tool.

A communication device may be any type of device capable of communication. For instance, a communication device may be a smartphone, tablet, laptop, desktop, server, personal digital assistant, wearable (e.g., smartwatch, glasses, etc.), or any other type of device.

In some embodiments, a local communication device 115 may be supported by a medical console 140. The local communication device may be permanently attached to the medical console, or may be removable from the medical console. In some instances, the local communication device may remain functional while removed from the medical console. The medical console may optionally provide power to the local communication device when the local communication device is attached to (e.g., docked with) the medical console. The medical console may be mobile console that may move from location to location. For instance, the medical console may include wheels that may allow the medical console to be wheeled from location to location. The wheels may be locked into place at desired locations. The medical device may optionally comprise a lower rack and/or support base 147. The lower rack and/or support base may house one or more components, such as communication components, power components, auxiliary inputs, and/or processors.

The medical console may optionally include one or more cameras 145, 146. The cameras may be capable of capturing images of the patient 118, or portion of the patient (e.g., surgical site). The cameras may be capable of capturing images of the medical devices. The cameras may be capable of capturing images of the medical devices as they rest on a tray, or when they are handled by a medical personnel and/or used at the surgical site. The cameras may be capable of capturing images at any resolution, such as those described elsewhere herein. The cameras may be used to capture a still images and/or video images. The cameras may be capturing images in real time.

One or more of the cameras may be movable relative to the medical console. For instance, one or more cameras may be supported by an arm. The arm may include one or more sections. In one example, a camera may be supported at or near an end of an arm. The arm may include one or more sections, two or more section, three or more sections, four or more sections, or more sections. The sections may move relative to one another or a body of the medical console. The sections may pivot about one or more hinge. In some embodiments, the movements may be limited to a single plane, such as a horizontal plane. Alternatively, the movements need not be limited to a single plane. The sections may move horizontally and/or vertically. A camera may have at least one, two, three, or more degrees of freedom. An arm may optionally include a handle that may allow a user to manually manipulate the arm to a desired position. The arm may remain in a position to which it has been manipulated. A user may or may not need to lock an arm to maintain its position. This may provide a steady support for a camera. The arm may be unlocked and/or re-manipulated to new positions as needed. In some embodiments, a remote user may be able to control the position of the arm and/or cameras.

In some embodiments, one or more cameras may be provided at the second location. The one or more cameras may or may not be supported by the medical console. In some embodiments, one or more cameras may be supported by a ceiling 160, wall, furniture, or other items at the second location. For instance, one or more cameras may be mounted on a wall, ceiling, or other device. Such cameras may be directly mounted to a surface, or may be mounted on a boom or arm. For instance, an arm may extend down from a ceiling while supporting a camera. In another example, an arm may be attached to a patient's bed or surface while supporting a camera. In some instances, a camera may be worn by medical personnel. For instance, a camera may be worn on a headband, wrist-band, torso, or any other portion of the medical personnel. A camera may be part of a medical device or may be supported by a medical device (e.g., endoscope, etc.). The one or more cameras may be fixed cameras or movable cameras. The one or more cameras may be capable of rotating about one or more, two or more, or three or more axes. The one or more cameras may include pan-tilt-zoom cameras. The cameras may be manually moved by an individual at the location. The cameras may be locked into position and/or unlocked to be moved. In some instances, the one or more cameras may be remotely controlled by one or more remote users. The cameras may zoom in and/or out. Any of the cameras may have any of the resolution values as provided herein. The cameras may optionally have a light source that may illuminate an area of interest. Alternatively, the cameras may rely on external light source.

Images captured by the one or more cameras 145, 146 may be analyzed as described further elsewhere herein. The video may be analyzed in real-time. The videos may be sent to a remote communication device. This may allow a remote use to remotely view images captured by the field of view of the camera. For instance, the remote user may view the surgical site and/or any medical devices being used. The remote user may be able to view the medical personnel. The remote user may be able to view these in substantially real-time. For instance, this may be within 1 minutes or less, 30 seconds or less, 20 seconds or less, 15 seconds or less, 10 seconds or less, 5 seconds or less, 3 seconds or less, 2 seconds or less, or 1 second or less of an event actually occurring.

This may allow a remote user to lend aid or support without needing to be physically at the first location. The medical console and cameras may aid in providing the remote user with the necessary images and information to have a virtual presence at the first location. In some embodiments, multiple remote users may be able to lend aid or support without needing to be physically at the first location. The multiple users may provide aid or support simultaneously or in sequence. A local communication device may be capable of communicating with multiple remote communication devices simultaneously.

The video analysis may occur locally at the first location 110. In some embodiments, the analysis may occur on-board a medical console 140. For instance, the analysis may occur with aid of one or more processors of a communication device 115 or other computer that may be located at the medical console. In some instances, the video analysis may occur remotely from the first location. In some instances, one or more servers 170 may be utilized to perform video analysis. The server may be able to access and/or receive information from multiple locations and may collect large datasets. The large datasets may be used in conjunction with machine learning in order to provide increasingly accurate video analysis. Any description herein of a server may also apply to any type of cloud computing infrastructure. The analysis may occur remotely and feedback may be communicated back to the console and/or location communication device in substantially real-time. Any description herein of real-time may include any action that may occur within a short span of time (e.g., within less than or equal to about 10 minutes, 5 minutes, 3 minutes, 2 minutes, 1 minute, 30 seconds, 20 seconds, 15 seconds, 10 seconds, 5 seconds, 3 seconds, 2 seconds, 1 second, 0.5 seconds, 0.1 seconds, 0.05 seconds, 0.01 seconds, or less).

In some embodiments, medical personnel may communicate with one or more remote individuals. The medical personnel may communicate with a single type or category of remote individuals, or with multiple types of remote individuals.

A second location 120 may be any location where a remote individual 127 is located. The second location may be remote to the first location. For instance, if the first location is a hospital, the second location may be outside the hospital. In some instances, the first and second locations may be within the same building but in different rooms, floors, or wings. The second location may be at an office of the remote individual. A second location may be at a residence of a remote individual.

A remote individual may have a remote communication device 125 which may communicate with a local communication device 115 at the first location. Any form of communication channel 150 may be formed between the rep communication device and the location communication device. The communication channel may be a direct communication channel or indirect communication channel. The communication channel may employ wired communications, wireless communications, or both. The communications may occur over a network, such as a local area network (LAN), wide area network (WAN) such as the Internet, or any form of telecommunications network (e.g., cellular service network). Communications employed may include, but are not limited to 3G, 4G, LTE communications, and/or Bluetooth, infrared, radio, or other communications. Communications may optionally be aided by routers, satellites, towers, and/or wires. The communications may or may not utilize existing communication networks at the first location and/or second location.

Communications between rep communication devices and local communication devices may be encrypted. Optionally, only authorized and authenticated rep communication devices and local communication devices may be able to communicate over a communication system.

In some embodiments, a remote communication device and/or local communication device may communicate with one another through a communication system. The communication system may facilitate the connection between the remote communication device and the local communication device. The communication system may aid in accessing scheduling information at a health care facility. The communication system may aid in presenting, on a remote communication device, a user interface to a remote individual about one or more possible medical procedures that may benefit from the remote individual's support.

A remote individual may be any user that may communicate remotely with individuals at the first location. The remote individual/user may lend support to individuals at the first location. For instance, the remote individual may support a medical procedure that is occurring at the first location. The remote user may provide support for one or more medical products, or provide advice to one or more medical personnel.

In some embodiments, the remote user may be a vendor representative. Medical products may be provided by one or more vendors. Typically, vendors may make arrangements with health care facilities to provide medical products. Vendors may be entities, such as companies, that manufacture and/or distribute medical products. The vendors may have representatives that may be able to provide support to personnel using the medical devices. The vendor representatives (who may also be known as product specialists or device reps), may be knowledgeable about one or more particular medical products. Vendor representatives may aid medical personnel (e.g., surgeons, surgical assistants, physicians, nurses) with any questions they may have about the medical products. Vendor representatives may aid in selection of sizing or different models of particular medical products. Vendor representatives may aid in function of medical products. Vendor representatives may help a medical personnel use product, or troubleshoot any issues that may arise. These questions may arise in real-time as the medical personnel are using a product. For instance, questions may arise about a medical product while a surgeon is in an operating room to perform a surgery. Traditionally, vendor representatives have been located at the first location with the medical personnel. However, this can be time consuming since the vendor representative will need to travel to the location of the medical procedure. Secondly, the vendor representative may be present but the vendor representative's help may not always be needed, or may be needed for a very limited time. Then, the vendor representative may have to travel to another location. It may be advantageous for a vendor representative to communicate remotely as needed with personnel at the first location. Thus, in systems and methods provided herein, the vendor representative may be a remote individual at a second location who may provide support remotely.

The remote users may be any other type of individual providing support, such as other medical personnel (e.g., specialists, general practice physicians, consultants, etc.), or technical support. Any description herein of vendor representatives may also apply to any other type of individual providing support, and vice versa.

In some embodiments, information about communications between remote users, such as vendor representatives, and the medical console (or any other device at the first location) may be collected and used for any of the processes described elsewhere herein. For instance, call data records may include one or more of the following: call start time, call end time, call duration, the identity of the individuals on the call (e.g., remote user identity, medical personnel identity such as identity of medical practitioner used to log into a medical console or the identities of all medical personnel present at a medical procedure), identity of the medical console making a call (e.g., each medical console may have a unique or semi-unique identity, which may or may not encode a health care facility identity and/or medical personnel identity), bandwidth on audio and video throughout the call, or any other factors. In some embodiments, factors, such as video or audio bandwidth may be indicative of the amount of activity that has occurred on the call. This may be indicative of the degree of active support provided by the vendor representative during the call.

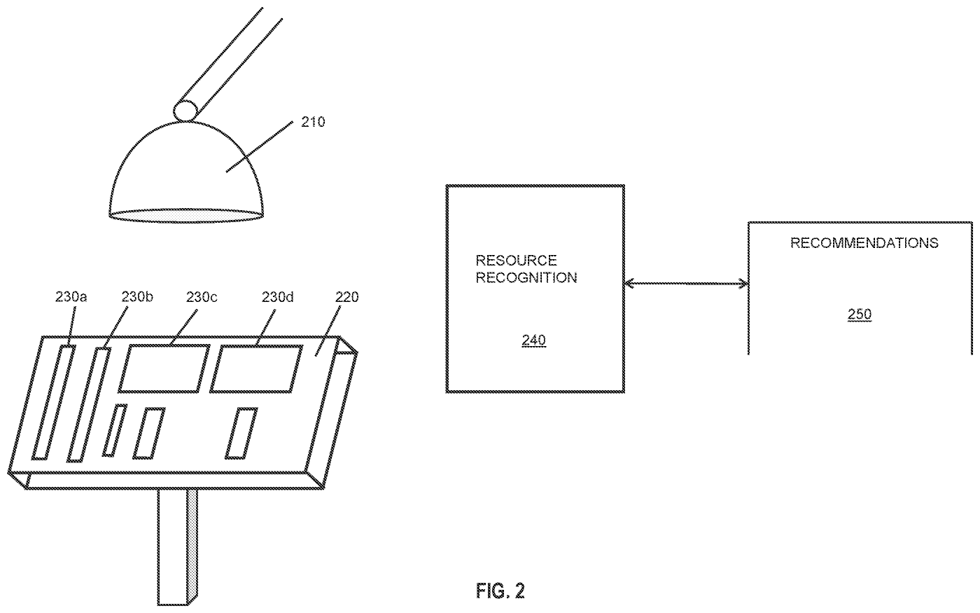

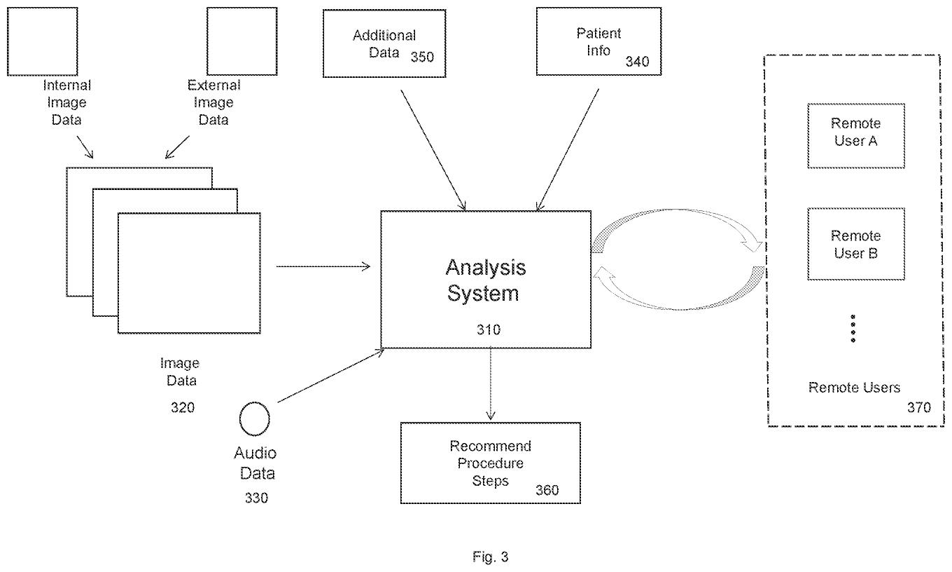

FIG. 2 shows an example of medical resources that may be recognized using a video capture system, in accordance with embodiments of the invention.

As previously described, one or more cameras 210 may be provided at a location of a medical procedure. The one or more cameras may include cameras on a medical console, supported on a ceiling, a boom, an arm, a wall, furniture, worn by medical personnel, or any other location. Multiple cameras may optionally be provided. The video collected by the cameras may be aggregated and/or analyzed by a video analysis system.

The one or more cameras may individually or collectively capture images of the medical resources. For example, medical resources may include medical products 230a, 230b, 230c, 230d, 230e that may be used at the location. In one example, one or more cameras may individually or collectively capture an image of medical products that may be provided at a single location, such as a tray 220.

The video analysis system may be able to recognize the medical products that are provided. The medical product may be recognized in accordance with medical product type (e.g., stent), or may be recognized specifically to the model level (e.g., Stent Model ABCD manufactured by Company A). In some embodiments, the medical products may have graphical codes, such as QR codes, barcodes (e.g., 1D, 2D, 3D barcodes), symbols, letters, numbers, characters, shapes, sequences of lights or images, icons, or any other graphical code that may be useful for identifying the medical product. The cameras may capture images of the graphical codes, which may be useful for identifying the product type, specific product model, and/or specific product (e.g., tracked to the individual product, or batch/group).

The medical resources may include individuals who may be present at a procedure, such as medical personnel. For example, the videos may capture images of the medical personnel during the procedure. In some embodiments, facial recognition, gesture recognition, gait recognition, or other video analysis may occur to recognize the identity of the individuals present, and/or actions taken by the individuals.

The medical resources may include location of the medical procedure. The medical console may be given a location identifier when the medical console is used. One or more video cameras may have a location identifier. In some instances, features, words, symbols at the location may be recognized to recognize the room location. In some instances, one or more GPS signals may be used to determine the location.

In some embodiments, audio information may be collected as well. For example, speech by medical personnel may be analyzed to detect words that may refer to medical products and/or usage thereof. In some instances, the sound of medical products being used may be analyzed and recognized. Medical products may have a unique or substantially unique audio signature when in use. In some instances, a frequency or degree of use or other type of usage specifics may be detected based on audio information. In some embodiments, the location of medical products may be discerned based on audio information. The audio system may be used to discern whether a product is outside or within a patient. The audio information may be analyzed independently or together within image information.

Medical records, surgeon prep cards, inputs by medical personnel, or any other sources may be used in recognizing the medical resources, such as medical products and personnel that are provided at a procedure.

Additionally, the systems and methods provided herein (video, audio, records, prep cards, inputs, etc.) may be used to track usage of the medical resources. For instance, the video may capture medical personnel lifting a medical product (e.g., from an instrument tray) and using it at a step during the procedure. The systems and methods provided herein may be able to recognize different steps of the procedure. The steps of the procedures may be predicted or known. In some instances, the steps of the procedure may provide context in trying to determine whether a particular medical product is being used. For example, if it is determined that a particular step is occurring, and that the step would require the use of a particular instrument, then the product that is imaged as being used may be interpreted within that context.

The timing and details regarding the actual use of the medical product may be recognized. Support given by a vendor representative at that time may also be recognized. In some embodiments, the timing and steps taken during the procedure may be used to determine efficacy of the product and/or support.

In some embodiments, the information may be collected passively without requiring any specialized input by medical personnel. For example, the images of the products may be automatically calculated and recognized.

Alternatively or in addition, medical personnel may provide some input or perform an action that may aid in detecting the resources (e.g., products) provided and/or used. In some instances, medical personnel may speak about the products that they are using. For example, as a medical personnel performs a step, the medical personnel may include information about the step and/or the product that is being used. One or microphones may connect information and be able to translate the speech into text and/or recognize the products described.

In another example, medical personnel may scan the medical products to be used. For example, they may use a scanner to scan one or more graphical code provided on the product. This may occur prior to the medical procedure or at the beginning of the medical procedure. In some instances, scanning may occur as products are used as well to track the use of the products. In some cases, one or more imaging devices may be used to scan the medical products.

Optionally, the devices or wrappers for the devices may include RFID or other type of near field communication. One or more scanners or readers may be provided to detect the communications coming from the device to recognize product usage.

The resources may be recognized using an analysis system 240. Based on the recognition, one or more recommendations 250 may be provided. The recommendations may be for medical resources to be used during the procedure. For example, specific products or medical personnel may be recommended. The recommendations may be made for the procedure, such as particular steps or techniques to use during the procedure. Such recommendations may be provided prior to a procedure, during a procedure, or after a procedure has been completed for future procedures.

The video capture and analysis systems may also capture images of the patient. The images of the patient may be analyzed prior to, during, or after a medical procedure. In some instances, the images of the patient may be analyzed to provide recommendations prior to, during, or after the medical procedure. For instance, steps for the medical procedure may be recommended. Specific techniques or products used may be recommended. Conditions of the patient may be monitored, and recommendations may be modified or maintained based on the condition of the patient. Conditions of the patient may include vitals for the patient, anatomical features of the patient, demographics of the patient, auxiliary inputs relating to the patient, detected visual features on or within the patient, response of the patient to steps performed during the procedure, or any other conditions.

In the systems and methods provided herein, an analysis system may gather information collected at one or more locations (e.g., first locations). The analysis system may gather information from multiple medical consoles or locations within a health care facility. The analysis system may gather information from multiple health care facilities. The analysis system may utilize video information, audio information, information from instruments that may be connected to a medical console, or information input by one or more medical personnel.

In some embodiments, the systems of the present disclosure may comprise a medical resource intelligence system that is configured to receive, process, update, and/or manage inventory information and/or tool usage information. In some cases, the medical resource intelligence system may be configured to manage and/or update the inventory information and/or the tool usage information based on an analysis of the images, video, or audio captured for a procedure (e.g., a medical procedure or a surgical procedure). As used herein, inventory information may comprise information on what types of medical tools, instruments, devices, or resources were previously available, are currently available, or will be available at some point in time. Inventory information may further comprise information on the quantities and availability of such tools, instruments, devices, or resources at different points in time, as well as information on when such tools, instruments, devices, or resources are expected to be used, depleted from stock, or received in a new order or shipment of orders. In some cases, inventory information may comprise information on a historical or projected usage of various tools, instruments, devices, or resources within a certain time frame, or with respect to a particular type of medical procedure, or with respect to a particular doctor, physician, surgeon, or other medical worker. As used herein, tool usage information may comprise information on what types of tools, instruments, devices, or resources have been used, are currently being used, or will be used in the future. In some cases, tool usage information may comprise information on how many tools have been used, are currently in use, or are expected to be used within a certain time frame. In some cases, tool usage information may comprise information on how long the tools have been used or will be used. In some cases, tool usage information may comprise information on what types of tasks or procedures have been completed or will be completed using the tools at some point in time. Tool usage information may correspond to usage of tools that were previously available in inventory, are currently available in inventory, or are expected to be available in inventory at some point in time in the future.

In some cases, the medical resource intelligence system may be configured to update or track inventory information based on the tool usage information. For example, the medical resource intelligence system may be configured to update or track inventory information based on a doctor's or surgeon's usage of one or more tools during a medical procedure, based on the preparation of the one or more tools for an upcoming medical procedure, or based on an expected use of one or more tools by a particular doctor or surgeon (e.g., based on a tool preference of the doctor or surgeon). The medical resource intelligence system may be configured to track a usage of one or more tools provided in an operating room (e.g., in a tool tray or a tool cabinet), detect what tools or in the tool tray or tool cabinet have been used or are being used (e.g., based on an optical or image-based detection of the usage of such tools), and update inventory information based on the detected use of the one or more tools. In some cases, tool usage may be detected based on a reading or a scan of one or more identifying features associated with or provided on the tool. The one or more identifying features may comprise, for example, a barcode, a quick response (QR) code, or any other visual pattern or textual data (e.g., alphanumeric sequence). In some cases, tool usage may be detected based on one or more images or videos captured using a camera or imaging sensor located in the operation room. The one or more images or videos may show a usage or a preparation of the tools by a doctor, a surgeon, or other medical worker or assistant before, during, and/or after one or more steps of a surgical procedure. In other cases, tool usage may be detected using a radio-frequency identification (RFID) tag associated with the one or more tools.

In some cases, the medical resource intelligence system may be configured to update tool usage information based on a doctor's or surgeon's usage of one or more tools during a medical procedure, or based on the preparation of the one or more tools for an upcoming medical procedure. The medical resource intelligence system may be configured to track a usage of one or more tools provided in an operating room (e.g., in a tool tray or a tool cabinet), and to determine what tools or in the tool tray or tool cabinet have been used or are being used based on an optical or image-based detection of the usage of such tools. In some cases, the optical or image-based detection may comprise identifying the tool based on one or more images or videos captured using a camera or imaging sensor located in the operation room. In some cases, the optical or image-based detection may comprise identifying the tool based on an optical reading or scan of one or more identifying features associated with or provided on the tool. The one or more identifying features may comprise, for example, a barcode, a quick response (QR) code, or any other visual pattern or textual data (e.g., alphanumeric sequence). In some cases, the medical resource intelligence system may be configured to track a usage of one or more tools provided in an operating room (e.g., in a tool tray or a tool cabinet), and to determine what tools in the tool tray or tool cabinet have been used or are being used, based on a radio-frequency identification (RFID) tag associated with the one or more tools.

In some cases, inventory information and/or tool usage information can be updated based on an interaction between a surgeon or medical worker and one or more tools provided in a tool tray or a tool cabinet. The interaction may comprise the surgeon or medical worker lifting a tool from the tool tray, placing the tool back down on the tool tray, repositioning or reorienting the tool relative to the tool tray, adding one or more tools to the tool tray, removing one or more tools from the tool tray, or replacing one or more tools on the tool tray. The inventory information and/or tool usage information can also be updated based on a number of times a tool has been lifted from the tool tray, or a length of time during which the tool is not in contact with the tray (e.g., when the tool is in use by a doctor, a surgeon, a medical worker, or a medical assistant).

In some cases, tool preferences of the surgeon or the healthcare facility for a particular type of procedure may be used to update inventory information or tool usage information. For example, if the surgeon or healthcare facility has a preference for a certain set of tools to be used during one or more steps of a surgical procedure, such preference may be used to update tool usage information or expected tool usage information for one or more upcoming surgical procedures, or for one or more upcoming steps for a surgical procedure. Further, such preference may be used to update inventory information. For example, if a surgeon having a particular tool preference has a procedure scheduled for a certain date, the medical resource intelligence system can update the inventory information based on that surgeon's particular tool preferences. In some cases, the medical resource intelligence system can update the inventory information based on an expected or predicted tool usage. Such expected or predicted tool usage may be determined in part based on the tool preferences of a particular surgeon or a particular healthcare facility in which a medical procedure is to be performed.

In some cases, the tool preferences for a particular surgeon may be determined based on a preference card of the surgeon. In other cases, the tool preferences for a particular surgeon may be determined based on one or more inputs, responses, or instructions provided by the surgeon. In some instances, the tool preferences for a particular surgeon may be determined based on a historical trend or usage of one or more tools by the surgeon for a particular type of surgery.

In some cases, inventory information and tool usage information may be used to determine which tools are in short supply, how many of such tools are in stock, and how many medical procedures can be supported or completed using those tools still available. The medical resource intelligence system may be configured to use the inventory information and/or tool usage information to place or queue an order for one or more additional tools or replacement tools. The medical resource intelligence system may be further configured to use the inventory information and/or tool usage information to provide one or more messages or alerts to a surgeon or a healthcare facility indicating the available stock for one or more tools, and which of the one or more tools are in short supply. In other cases, inventory information and tool usage information may be used to determine which tools are well stocked, how many of such tools are in stock, and how many medical procedures can be supported or completed using those tools currently available. In some cases, the medical resource intelligence system may be configured to use the inventory information and/or tool usage information to order, preorder, or reorder one or more tools based on an expected need for the one or more tools in an upcoming surgical procedure.

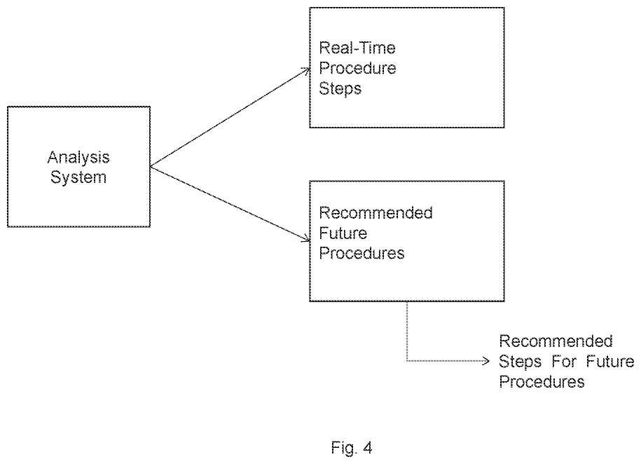

FIG. 3 shows an example of how video captured may be utilized by an analysis system in order to recommend medical procedure steps, in accordance with embodiments, of the invention.

An analysis system 310 may gather data that may be useful for generating one or more recommended medical procedure steps. An analysis system may employ a computer system as described elsewhere herein. An analysis system may comprise one or more processors that may individually or collectively execute one or more steps as provided herein. The analysis system may comprise one or more memory storage units comprising non-transitory computer readable media that may comprise code, logic, or instructions for performing any of the steps provided herein.

Various type of data may be provided to the analysis system. For example, image data 320 may be provided to the analysis system. Image data may be generated with aid of a video capture system as described elsewhere herein. Image data may be collected prior to, during, or after a procedure. The image data may be captured with aid of one or more cameras having the characteristics as described elsewhere herein.

In some embodiments, the image data may comprise internal images and/or external images. For example, the internal images may include images internal to a patient. For instance, the images may include images of a surgical site. The images may include endoscopic or laparoscopic images. Internal images may include images that are internal to the patient body. In some instances, one or more cameras may be positioned within the patient's body. In some instances, external images may be provided. External images may include images external to a patient. For instance, the images may include images of a patient's body from outside the body, or an image of the location where a procedure is taking place. In some instances, only internal images may be provided, only external images may be provided, or both internal and external images may be provided and/or analyzed by the analysis system. The internal images and/or external images may be interfaced with a medical console. Optionally, one or more internal images and/or external images may be provided to the analysis system without needing to interface with the medical console.

In another example, audio data 330 may be provided to the analysis system. One or more microphones may be provided at a location where a procedure is taking place. In some embodiments, one or more microphones may be provided on or supported by a medical console. Optionally, one or more microphones may be provided external to the medical console.

Optionally, patient information 340 may be provided to the analysis system. Patient information may include medical records, medical history, inputs provided by medical personnel, information provided by the patient, or any other information. In some instances, patient information may include patient medical data, data from previous hospitalizations or clinic visits, laboratory test results, imaging results, family medical history, nutrition information, exercise information, demographic information (e.g., age, weight, height, race, gender, etc.) or any other information pertaining to the patient.

In some embodiments, additional information 350 may be provided to the analysis system. In some instances, the additional information may include information from one or more auxiliary sources that may be collected prior to, during, or after the medical procedure. In one example, auxiliary sources may include one or more additional instrument or medical device that may be able to collect information about the patient. The auxiliary sources may be connected to the medical console and/or provide data to the medial console. For example, a medical console may comprise one or more input ports to which one or more auxiliary devices may be connected. Examples of auxiliary devices may include, but are not limited to, endoscopes, electrocardiogram (ECG) devices, laparoscopes, oximeter, or any other type of device. The data from the auxiliary sources may be analyzed and/or provided to one or more remote users 370.

The analysis system may make recommendations based on the data received. For instance, the analysis system may recommend one or more steps for a medical procedure 360. For example, a medical procedure may comprise one or more steps. A step may comprise one or more levels of sub-steps. The steps may include information about actions to be taken by medical personnel, medical techniques, patient anatomy, and/or recommended products for particular actions.

The analysis system may receive information prior to a medical procedure and may optionally make recommendations prior to the medical procedure. Medical personnel may be able to review the recommendations prior to the medical procedure. The medical personnel may or may not choose to follow the recommendations.

The analysis system may receive information during a medical procedure. For example, images and/or audio collected during a medical procedure may affect recommendations that are provided during a medical procedure. For example, prior to a medical procedure, there may optionally be a set of recommended steps. One or more steps may be maintained or modified based on information that is collected during the medical procedure. The recommendations may be updated in substantially real-time as data is collected and provided to the analysis system. Even if an initial recommendation is not provided prior to a medical procedure, the data collected may allow recommendations to be formulated during the medical procedure. This may allow the system to advantageously adapt the recommendations based on patient condition and/or data collected during the procedure. For example, based on data collected during Step 10, Recommended Step 11 may change.

Optionally, an analysis system may receive data after a medical procedure. The data may be collected while the patient is at the first location immediately after the procedure. The data may be collected while the patient is in post-surgery recovery. One or more recommendations may be formulated based on data collected after the surgery as well. The recommendations may be provided for future surgeries of similar type. The recommendations may be provided to the medical personnel to show how the procedure may have been performed differently to yield different outcomes. Collecting data post-procedure may allow for a better sense of patient outcome after procedure which may be valuable data for analyzing how the procedure was conducted and recommendations for future procedures. These may include procedures that are coming up within any timeframe (e.g., within the next hour, days, months, or years, etc.). This may refer to future procedures for the same patient or other patients.

Recommendations provided by the analysis system may be viewed by medical personnel that are present for the medical procedure. In some instances, the recommended steps may be streamed to an external display at a location of the procedure. For example, a display on a medical console or separate from a medical console may show the recommended steps. The recommendations provided by the analysis system may be viewed by one or more remote users 370. In some embodiments, support may be provided by a single remote user or multiple remote users. Remote users may be able to view information simultaneously and provide feedback. In some instances, local medical personnel and/or one or more remote users may view the recommendations and choose to agree or disagree with the recommendations. For example, one or more remote users may provide feedback regarding the recommended steps and may suggest modifications to the recommended steps provided by the analysis system. For example, allowing viewing by remote users may allow one or more (e.g., multiple) experts to view and confirm the next steps or modify the next steps based on real-time feedback. This may allow for medical personnel to be supported in an efficient manner—the recommended steps may be viewed by all parties in real-time and subsequent feedback and modifications/updates may also be viewed in real-time by the various parties. This may advantageously allow for real-time collaboration between multiple parties.

FIG. 4 shows an example of various types of procedure recommendations that may be formulated by a video analysis system, in accordance with embodiments of the invention.

As previously described, the analysis system may provide real-time procedure recommendations. This may include recommended steps for a procedure that is currently taking place or that is imminent (e.g., being prepped for). For example, for a procedure, the system may recommend Step A, Step B, Step C, etc. As data is collected before or during the procedure, the steps may optionally be modified in real-time. In some instances, as one or more remote users provide feedback, the steps may also be modified in real-time. For example, based on image or audio data collected during Step B, Step C may be modified to Step C′, and Step D may be modified to Step D′. The number of steps, or recommendations for products used during the steps may vary based on data collected in real-time. Medical personnel may be able to view the changes in steps in real-time which may allow them to make preparations in real-time. For example, if a newly recommended step requires the use of a medical product that was not already prepared, one or more medical personnel can prep the medical product so that it will be ready when needed.

The analysis system may also provide recommendations for future procedures. The recommendations may be provided for future procedures for the same patient, or for other patients. The analysis systems may be providing recommendations for imminent procedures (e.g., already know that Patient X will have a surgery next week). The analysis system may also be providing recommendations for future procedures if/when they occur (e.g., after Procedure A, there may typically be a Procedure B to follow-up in several years, etc.). The analysis system may make recommendations on timing and/or types of future procedures that may be likely based on the data collected during the procedure and/or other information. The analysis system may make recommendations based on data that may be collected post-procedure and/or various patient outcomes. In some instances, data from clinical follow-up visits may be analyzed to make a recommendation. For example, after a procedure, a patient may visit a clinician one or more times. Based on data gathered during the clinical visits, a follow-up procedure may be recommended. The timing for the follow-up procedure may be recommended.

For the future procedures, recommended procedure steps may be provided. For future procedures, the recommended steps may optionally be provided in the same level of detail or a broader level of detail than procedures that are imminent (e.g., that are being prepped for) or that are currently taking place. In some instances, one or more medical personnel may view the steps for the future procedures and provide recommendations or modifications.

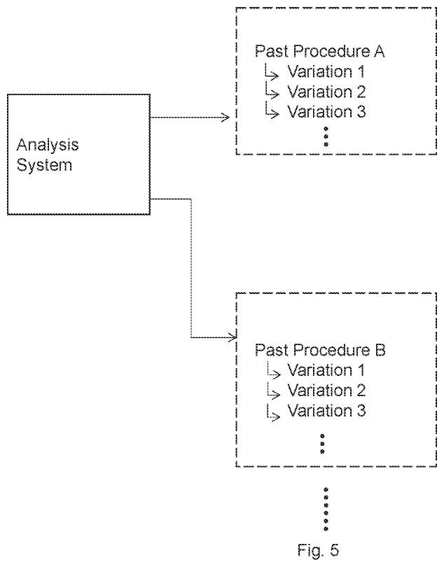

FIG. 5 shows an example of past procedure analysis and variation recommendations, in accordance with embodiments of the invention. The analysis system may receive information about one or more completed procedures. For example, the analysis system may receive information about completed procedures relating to a single patient or to multiple patients. The analysis system may receive information from a large data set of the same type or similar types of procedures, or procedures that may be used to treat a similar condition. The various data sets may include data from multiple procedures at the same health care facility. The various data sets may include data from multiple procedures across multiple health care facilities. The analysis system may advantageously collect data from multiple health care facilities relating to various procedures. The data may be compliant with privacy rules or regulations. The data may be HIPAA-compliant. The data collected may include any of the type of data as described elsewhere herein, including but not limited to, image data, audio data, patient data, patient outcomes, or additional information.

The analysis system may analyze a past procedure. Variations to the past procedure may be recommended based on past information and patient outcomes. For example, if multiple steps occurred during a past procedure, variations to the procedure may include removing steps, adding steps, changing the order of steps, and/or modifying steps. Step details may be modified, which may include the actions taken by the medical personnel, products that may be used for such actions/steps, identities of medical personnel that may perform the steps, timing of steps, various techniques that may be implemented, or any other factors.

For example, for a Past Procedure A, one or more recommendations may be presented (e.g., Variation 1, Variation 2, Variation 3, etc.). The variations may be independent of one another. The variations may be designed to be performed separately from one another. Alternatively, one or more variation may be combined. The variations may be presented that would likely improve patient outcome. In some instances, the variations may be presented that would likely procedure any type of desired outcome. Examples of factors of a desired outcome may include improved patient outcome (e.g., overall recovery status, recovery time, reduction of complications), reduced procedure time, increased efficiency, reduced cost, etc.

In some embodiments, the variations may be ranked or presented in order. The variations may be ranked in accordance with desired outcome. The one or more factors relating to the desired outcome (e.g., improved patient outcome, increased efficiency, etc.) may optionally be weighted in determining the ranking for the variations. In some instances, a quantitative or qualitative indicator of success or accuracy rate may be provided with each variation. For example, an expected value relating to a desired outcome (e.g., a score) may be displayed with each variation. In some instances, a general score may be presented. Alternatively or in addition, one or more scores relating to one or more factors may be presented with each variation (e.g., a patient health score, patient recovery time score, time reduction score, etc.). This may provide a viewer with some sense of how the different variations may change the outcome from the past completed procedure.

The rank, success, and accuracy rate may be determined based on the collected data sets of successful procedures of similar type and/or patients with similar conditions. The rank, success, and accuracy rate may be controlled by input/output parameters provided by one or more experts. For instance, one or more reviewers may provide input that may affect the recommendations and variations.

In some embodiments, the variations may be presented as text. For example, the variations may include words that may describe changes to the steps that are recommended for the procedure. In some instances, the variations may be presented as image and/or video. For example, still images or portions of video that may relate to the changes in the procedure may be displayed. In some instances, video may be taken from a portion of a procedure taken at another instance, and may be shown to demonstrate the variation in the step. The variation in the step may or may not be spliced into a video that shows the past completed video, or presented as a side-by-side comparison with a step that was completed in the past procedure but is now being modified. In some instances, audio, such as speech or sounds may be used to present the variations.

Variations may be provided for various past completed procedures. For example, Past Procedure B may also be presented with variations. A user may be able to access information about a past procedure and view possible variations. The variations may be ranked according to desired outcome. Any number of variations may be presented. For example, a threshold number of variations may be presented to a user. The threshold may be determined by the user, a health care facility, the analysis system or any other party. The number of variations presented may depend on the degree of improvement that is available. For example, of no variations are detected that would improve the desired outcome, then no variations may be presented. In some instances, if a larger number of variations are detected that would improve the desired outcome, then a larger number of variations may be presented. In some instances, the number of variations that are displayed may depend on the number of variations that improve the desired outcome by a threshold amount. The threshold level of improvement for desired outcome may be fixed or may be determined (e.g., by the user, a health care facility, the analysis system or any other party).

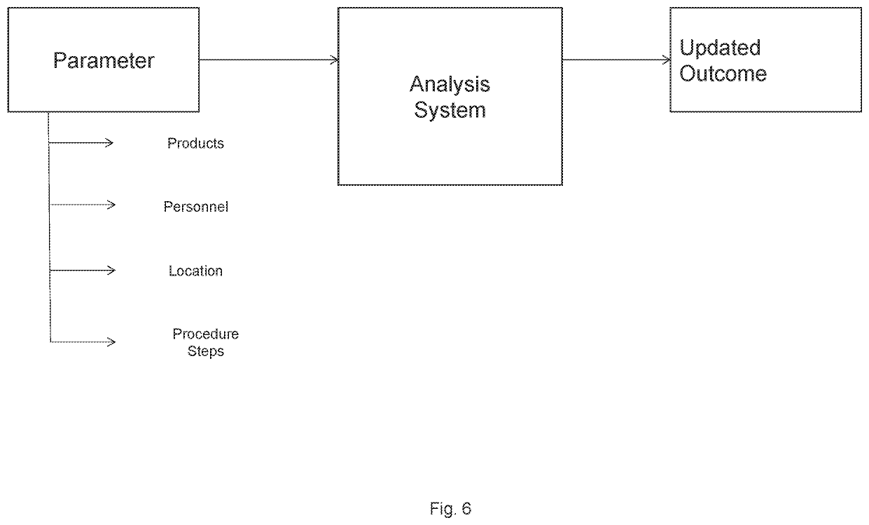

FIG. 6 shows an example of how various input parameters may affect an updated outcome by a video analysis system, in accordance with embodiments of the invention.

One or more procedure input parameters may be provided to an analysis system to predict an outcome for a procedure. The one or more input parameters may be provided by a user. For example, medical personnel, a health care facility administrator, a patient, a social worker, or any other user may be able to provide one or more input parameters. In some instances, original input parameters may be provided or suggested with aid of one or more processors. For example, one or more processors may automatically generate a set of input parameters. In some embodiments, one or more processors may automatically generate multiple sets of input parameters that may be used to compare potential procedure outcomes. In some instances, one or more sets of input parameters may be provided with aid of one or more processors and one or more users may adjust one or more of the suggested input parameters.

The input parameters may relate to any medical condition of a patient or any operating condition for a procedure. For example, the medical products used during a procedure may be provided as an input parameter. For example, a set of one or more medical products may be used during a medical procedure. The level of specificity may include a type of medical product (e.g., stent with certain specifications) or may include the specific brand and/or model of the product (Stent ABC manufactured by Company XYZ). In some instances, various functional equivalents of products may be considered, such as products models and/or manufacturers that may be capable of being used for similar functions or conditions. In one example, using Stent ABC by Company XYZ may show 10% improved outcomes over using Stent LNM from Company 123.

The input parameters may include identities of medical personnel. For example, different medical personnel may be involved during a procedure. This may include surgeons, physicians' assistants, nurses, or other individuals who may be present and/or involved with the procedure. For example, the system may be able to detect that Surgeon A typically has better outcomes than Surgeon B when performing certain types of procedures.

The personnel input parameter may also include identities of remote users. For example, vendor representative identities, specialist identities, tech support identities or other individuals who may provide remote support may be provided as parameters. For example, when a particular vendor representative provides support, outcomes may improve by 5%.

Another input parameter may include location of the procedure. The location of the procedure may refer to an identity of a health care facility. For example Hospital ABC may provide improved chances of a good outcome relative to Hospital DEF. In another example, the location of the procedure may include a specific room or region at a health care facility. For example, performing a particular type of procedure in Operating Room 17 may statistically improve one's chances over performing the same type of procedure in Operating Room 12. In another example, the type of location for performing the procedure may be analyzed. For example, using an operating suite with X specifications may yield different outcomes than using an operating suite with Y specifications (e.g., size, ventilation, lighting, instruments, temperature, etc.).

In another example, input parameters may include procedure steps. For example, for treating a particular patient condition, various types of procedures or techniques may be employed. For example, steps performed during the procedure may be varied. For example, at step 5 of the procedure, using technique A may yield different outcomes than using technique B. Various combinations of procedure steps may be compared.

Any of the input parameters described are provided by way of example only and are not limiting. Additional input parameters may be provided and/or compared as well. Various combinations of input parameters may be compared. The analysis system may receive and/or consider the input parameters and may provide information relating to a predicted outcome for the procedure.

In some instances, an input parameter module may generate the input parameters. The input parameter module may be part of the analysis system or may communicate with the analysis system. In some instances, an input parameter module may generate combinations of input parameters. For example, the input parameters may go through and generate numerous combinations of input parameters utilizing machine learning, as described elsewhere herein. The analysis system may employ machine learning as described elsewhere herein, to provide an outcome.

The analysis system may provide a predicted outcome based on input parameters. The predicted outcome may be for a procedure that has not yet taken place. For example, prior to performing a procedure, a user may wish to provide or compare input parameters to view a predicted outcome. For example, medical personnel may wish to consider using Step 5A instead of Step 5B during a procedure, and may view the predicted outcomes to help in coming to a decision on the steps to take. In a similar example, the medical personnel may wish to consider using Product ABC instead of Product MNL during a procedure and may wish to view the predicted outcomes to help in coming to a decision on which product to use.

The outcome may be for a procedure that is currently taking place. Even during a medical procedure, medical personnel may come to a point in which a decision may need to be made. The various possibilities may be compared to come to a real-time decision on the path to take. The outcome for the current procedure may be forecasted for the different paths that may be taken.

The outcome may be for a past procedure. For example, a past procedure may be analyzed to see how different input parameters could have yielded different outcomes. Various combinations of input parameters may be considered to determine possible different outcomes. In some instances, the outcomes may be ranked. Different parameters or combinations thereof that would have yielded the various outcomes may be presented. For example, a user may see that if a user had used Step 5A instead of Step 5B, the outcome would be different. The changes in parameters for the various outcomes may be presented. In some instances, the various parameter values that yielded the outcome may be presented in a visually associated manner with the outcome.

The outcomes may be presented in a quantitative and/or qualitative fashion. For example, the outcomes may provide qualitative statements about how outcomes may vary. For example, qualitative statements such as ‘less blood loss’, ‘faster recovery time’, ‘increased patient satisfaction’, ‘reduced patient pain’, etc may be provided. In some instances, quantitative data about the various outcomes may be provided. For example, ‘increased X.X% survival rate’, ‘Y% reduced recovery time’, ‘$Z cost’ or other types of quantitative information relating to the outcomes. The outcomes may be presented in a list or ranking, or any other manner.

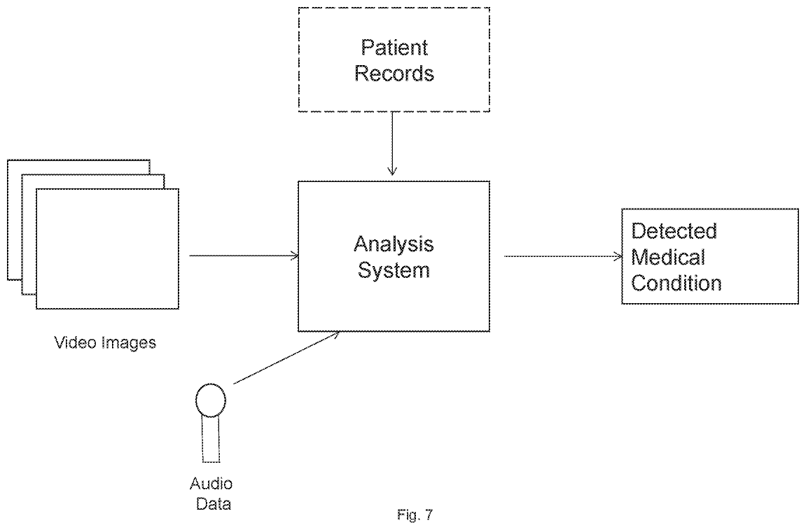

FIG. 7 provides an example of how a video analysis system may automatically detect a medical condition, in accordance with embodiments of the invention.

In some embodiments, an analysis system may receive data from one or more sources. For example, data from one or more video images, audio data, patient records, or any other type information (e.g., additional information) may be presented.

The video data may be any type of image data, as described elsewhere herein. For example, the video data may be captured with aid of a video capture system. The video capture system may have any characteristics as described elsewhere herein. The video capture system may comprise one or more auxiliary data sources, or video data from one or more video capture systems may be incorporated with data from one or more auxiliary data sources as video images. Images may be collected with aid of one or more internal cameras and/or external as described elsewhere herein. The cameras may be positioned external to a patient's body or may be positioned internally within a patient's body. The one or more cameras may collect images of the patient. The video data may include images of a surgical site of the patient, a region within the patient, an external surface of the patient, or any other image of the patient. The video data may comprise images at a location of the procedure.

Audio data may be captured and/or analyzed by the analysis system. For example, audio data may be collected with aid of one or more microphones. Audio data may be captured with aid of one or more auscultation devices.

Patient data and/or any additional data may be obtained and/or analyzed by the analysis system. Any types of patient data and/or additional data as described elsewhere herein may be incorporated.

The analysis system may analyze the data provided to provide a detected medical condition. The detected medical condition may be a condition that does or does not relate to a health condition for the patient for which a procedure may occur. For example, a patient may have health condition A, for which a procedure may occur. During the procedure, the analysis system may collect data that may be used to detect health condition B. Detected health condition B may have been previously unknown for the patient. The detected medical condition may have been previously unknown for the patient. In some instances, the detected medical condition may have been previously known for the patient, but the degree or progression of the detected medical condition may have been previously unknown for the patient. In another example, the detected medical condition may have been previously known for the patient, but may have been unrelated to the procedure taking place.

The detected medical condition may relate to any type of condition for the patient. The detected medical condition may be detrimental to the patient's health. The detected medical condition may or may not be neutral in relation to the patient's health. The detected medical condition may affect the expected life expectancy or quality of life of the patient. The detected medical condition may include a chronic condition, disease presence, disease progression, injury, trauma, cut, tumor, inflammation, infection, anatomical variation, or any other condition relating to the patient. The detected medical condition may or may not have urgent implications on the patient's health.

The detected medical condition may or may not affect recommended steps for the procedure. For example, a procedure may be taking place with respect to a patient, or imminently scheduled to take place, when the analysis system detects the medical condition. One or more recommended procedure steps may be provided for a medical procedure relating to the patient. When the medical condition is detected (during the procedure or prior to the procedure), there may be adjustments that may be made to the recommended procedure. For example, one or more steps may be removed, added, altered, or the order may be changed. Different medical products (e.g., tools) may be recommended. The medical condition may also be detected after a procedure has been completed. Recommendations for follow-ups or subsequent procedures may be made or altered based on the detection of the medical condition.

In one or more examples, video data may be analyzed to provide information relating to the detected medical condition. For example, the detected medical condition may be visually discernible in the video data captured by the video capture system. The medical condition itself may be visually detectable (e.g., presence of a tumor) or one or more visual indicator may be provided of a possible medical condition (e.g., swelling of a certain part may be indicative of a condition). In some instances, external indicators of a patient (e.g., bruising, discoloration, lesions, rashes, swelling, etc.) may be considered in detecting a medical condition. The visual indicator may be considered with additional information, such as patient records, to provide a likely medical condition.

In some instances, machine learning systems, as provided elsewhere herein, may be employed to analyze the data (e.g., video data, audio data, records) alone or in combination to detect a medical condition. For example, the systems and methods provided herein may analyze images captured. Object recognition techniques and/or pixel-based analysis may occur in order to detect and/or identify possible medical conditions.

The systems and methods provided herein may advantageously provide an early warning system of possible medical conditions. For example, if a patient is unaware of health condition B, but is undergoing a procedure for health condition A, the patient may be made aware of health condition B and may be able to take proactive action. Similarly, the recommendations to a procedure may be adjusted as needed based on the detected medical condition to provide improved patient outcomes. The systems and methods provided herein may permit for detection or early detection or identification of particular medical conditions, such as diseases, with added details of diagnosis or prognosis. The systems and methods provided herein may provide such information before a procedure, during a procedure, or after a procedure.

In some embodiments, an in-depth analysis may occur prior to a procedure, and the medical condition may be detected prior to the procedure. Recommendations relating to the procedure may be updated as needed. In some instances, a detected condition may result in the recommendation that a procedure be canceled, delayed, that different techniques or products be employed, or that different remote users provide support.

As previously described, the medical condition may be detected during the procedure. Recommendations relating to the procedure steps may be updated in real-time. Based on the detected condition, the ongoing procedure may or may not be altered. In some instances, recommendations may be made for actions to be taken after the procedure is completed.

Optionally, the medical condition may be detected after the procedure. For example, data may be collected relating to the patient after the procedure has been completed. The data may continue to be collected at the same location the procedure has occurred, immediately after the procedure. In some instances, the data may be collected post-surgery while the patient is at a recovery suite or other type of location. Upon detection of the medical condition, recommendations may be made for actions to be taken after the procedure is completed and/or for additional procedures or follow-up.

In some instances, medical personnel may be made aware of the detected medical condition in a real-time alert. For example, if the condition is detected while the patient is undergoing a procedure, a visual or audio alert may be provided to the medical personnel. Information about the detected medical condition may be provided on a medical console and/or a local communication device. Information regarding the detected medical condition may be displayed on any display device at the location of the procedure. In some instances, a patient's medical records may be updated with information about the detected medical condition. The patient's medical records may be automatically updated without requiring human intervention. In some embodiments, remote medical personnel may be made aware of the detected medical condition. For example, a patient's clinician (e.g., primary care provider) may be sent a message about a possible detected medical condition and asked to follow-up.

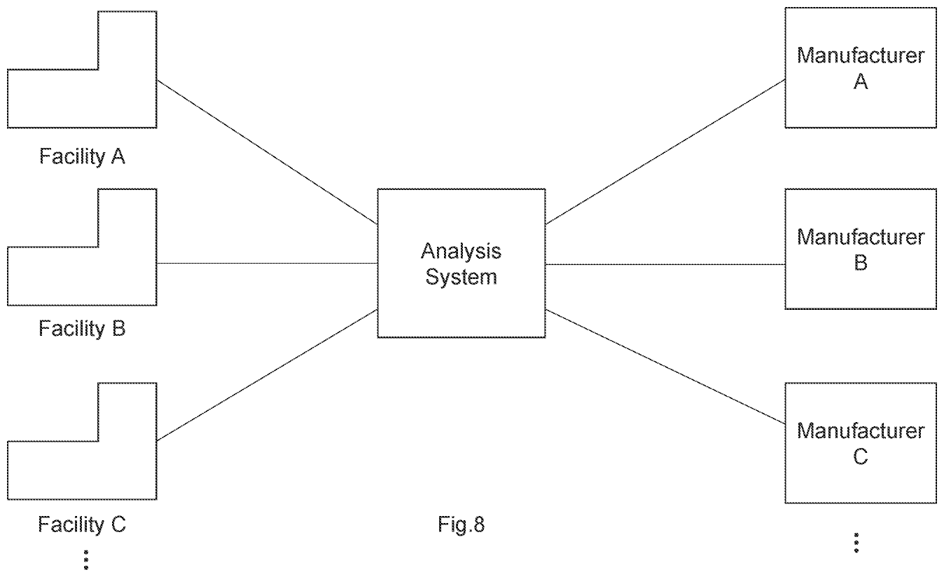

FIG. 8 provides an example of how various inputs from facilities may be used by the analysis system to provide recommendations to product manufacturers, in accordance with embodiments of the invention.

In some instances, data may be collected prior to, during, and/or after a procedure. The data may include video data, such as data captured with aid of one or more video capture systems. The video capture systems may have any characteristics as described elsewhere herein. The data may include audio data, patient records, or any other additional information. The data may include product usage information. The data may include information about cost for using or acquiring products.

The data may be provided from one or more health care facilities. For example information may be provided from a single health care facility and may be analyzed with respect to that health care facility. In other examples, the data may include information gathered from multiple health care facilities (e.g., Facility A, Facility B, Facility C, . . . ). Advantageously, a large data set may be collected relating to various procedures that may be undertaken using various medical products.

The analysis system may gather the collected data and make one or more recommendations relating to medical products. The recommendations may be made for particular medical products that may be used during one or more procedures. For example, usage data, patient outcomes, medical personnel feedback, or other type of information may be analyzed to make recommendations relating to one or more medical products.

Medical products may be made and/or sold by one or more manufacturers. Any reference herein to a manufacturer may include or incorporate any reference to one or more vendors. Product recommendations may be shared with one or more manufacturers. For instance, product recommendations for a particular existing product made by a particular manufacturer may be shared with that manufacturer. In some instances, such recommendations may be provided to such corresponding manufacturer only. In other instances, recommendations may be provided to manufacturers with similar products, or functionally equivalent products.

Recommendations regarding an existing medical product or functionally equivalent product may include information about how a medical product may be adjusted or modified. Such recommendations may be designed to yield improved functionality, better patient outcomes, higher usage rate of the medical product, more efficient procedure, lower of cost of manufacture, less waste, or any other result.

In some embodiments, recommendations for a product may include recommendations that are not tied to a particular existing medical product. The recommendations may be for a type of product that is similar to an existing product. In some instances, the recommendations may be for a type of product that does not have a functional equivalent or is not similar to an existing product, but that would allow a desired result, such as improved functionality, better patient outcomes, higher usage rate of the medical product, more efficient procedure, lower of cost of manufacture, less waste, or any other result. Such information may be conveyed to a single manufacturer or to multiple manufacturers. Such information may be conveyed to a manufacturer that has a functionally equivalent product or who has a product in a similar space or functionality.

The systems and methods provided herein may allow manufacturers to advantageously receive the benefits of access to a large data set about product usage. The analysis system may see how various products are tied to patient outcomes or other desired results and provide access to such information to manufacturers. The analysis system may advantageously make suggestions as to how products may be improved to yield more improved desired results and convey such information to manufacturers who may make the products. This may allow for multiple parties to advantageously use the information gain utilizing the video capture systems in order to improve products and improve desired results from a procedure.

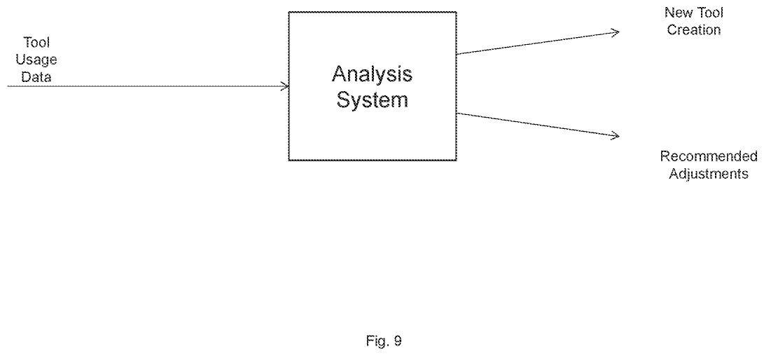

FIG. 9 shows an example of various recommendations that may be provided to a manufacturer in accordance with embodiments of the invention. As previously described, the analysis system may receive information relating to medical products, such as medical tools. Any description herein of a tool may apply to any other type of medical product and vice versa. The data may be captured with aid of a video capture system, audio system, medical personnel feedback or input, patient information, additional information, or any other data source.

The data may include information or specification about the tools themselves. For example, the data may include information about a tool's dimensions, materials in the tool, functionality, how the tool is used, time(s) at which the tool is used, and so forth.

Such information may optionally be provided by a manufacturer. For example, product specs may automatically be collected or sent by a manufacturer. A manufacturer may or may not choose to provide additional information about a product. A manufacturer may be presented with an option to provide additional information about the product. In some instances, third party public sources may be automatically searched (e.g., crawled) to find public information relating to a product.

In some instances, such information may be collected with aid of one or more video capture systems. The video capture systems may capture images of the medical products prior to, during, or after a procedure. The images collected by the video capture system may be analyzed to recognize the medical products. In some instances, object recognition techniques may be used to recognize the products. The object recognition techniques may recognize the type of medical product, or the exact brand or model of the product. Machine learning techniques may be used to recognize the medical product and/or correlate the medical product with an existing product. When an existing product is recognized, information about that product may be associated with the product whose image is captured. For example, if Stent Model ABC is recognized, then the specs relating to Stent Model ABC may be associated with the medical product that is captured by the video.

In some instances, machine vision systems may be used to directly recognize specifications relating to the medical product. For example, the dimensions to the medical product may be gathered based on the image of the medical product captured by the video. In some instances, a fiducial marker or any other reference marker may be provided for scale, or to aid in determining the dimensions. The shape of the medical product may be determined with aid of the video capture systems. In some instances, the potential materials for the medical product may be determined based on the images capture by the video system. In some embodiments, audio systems may be utilized for recognizing specifications relating to the medical product. The sound of the product in use may be used to recognize specifics of the product. In some instances, a product or type of product may have a unique sound signature when in use. Similarly, medical personnel may say the name or a characteristic of the product prior to or during the procedure.

The machine vision/audio systems may be used to directly recognize usage information relating to the medical product. For example, one or more cameras may capture images of the medical product as it is used. The motions relating to the medical product may be recognized. For example, the video capture systems may capture images of the medical product being picked up by a medical personnel. The video capture systems may capture images of the medical personnel using the product in relation to the patient. The motions of the medical personnel while using the product may be capture and/or analyzed. Motions of the medical personnel may be capture and/or analyzed. In some instances, the motions of the medical product and/or medical personnel may be analyzed within the context of steps taken for the procedure. One or more steps may be recognized based on the motions and recognized product. Similarly, audio information about the product may be collected and/or analyzed to provide usage information relating to the product. The sound (e.g., unique or substantially unique audio signature) of a product being used may indicate that the product is being used. In some instances, a level of use may be detected based on audio information. In some instances, the audio systems may be able to detect relative placement of the sound, such as location of origination of the sound. Medical personnel may also use words to describe use of the product.

Timing information relating to the usage of the medical product may be collected and/or tracked. For example, timing of when the medical product is used and/or for a step involving the medical product may be collected and/or analyzed. The timing of the product use may be detected using machine vision/audio systems. In some instances, if the measured time to perform to perform a step involving the medical product significantly exceeds an expected amount of time to perform the step, then the step may be flagged for further analysis. In some instances, the increased amount of time may be indicative that something did not go as expected, or that there was something wrong that occurred during the step. In some instances, medical products used during the step may be analyzed within the context that something may not have gone as expected. For example, when longer than expected steps occur regularly when a particular medical product is used, then recommendations may be made to improve or adjust the product to provide desired results.

The analysis system may make recommendations relating to a product based on the various data collected. The analysis system may utilize machine learning techniques, such as those described elsewhere herein, in recognizing the product, recognizing steps and/or usage of the product, and/or making recommendations with respect to the product.

The recommendations may include recommendations with respect to usage of an existing product. For example, one or more recommendations may be provided to use a particular model or brand of product for a particular type of procedure. Such recommendations may be generalized to all parties, may be specific to a health care facility, and/or may be specific to medical personnel.

For instance, when product X shows improved desired results with respect to product Y, regardless of context, then a generalized recommendation may be made for parties to use product X. Such recommendations may be provided prior to or during a procedure. The recommendations may be made with respect to procedure type.

In another instance, the performance of the products in yielding a desired result may be analyzed within the context of a health care facility. For example, data may be collected with respect to health care facilities. If at Facility A, Product X yields a more desired outcome than Product Y, while at Facility B, Product Y yields a more desired outcome than Product X, then at Facility A, Product X may be recommended while at Facility B, Product Y may be recommended. In some instances, health care facility preferences and rules may also be taken into account. For example, if Facility A has a deal with a manufacturer that makes Product Y, then Product Y may still be recommended over Product X. In some instances, the various factors to yield a desired outcome may be measured and considered. In some instances, one or more factors may be weighted. For instance, existing agreements between a facility and a manufacturer may be weighted along with patient outcome, efficiency, or other factors for the desired result.

In some instances, the performance of the products in yielding a desired result may be analyzed within the context of medical personnel. For instance, data may be collected with respect to different medical personnel. Medical personnel may have their own preferences or may have different results for the same product. For example, if Practitioner A achieves a more desired outcome with Product X than Product Y, and Practitioner B achieves a more desired outcome with Product X than Product Y, then Product X may be recommended for Practitioner A, and Product Y may be recommended for Practitioner B. Medical personnel preferences may or may not be taken into account when making these recommendations. In some instances, the recommended product may not be aligned with the medical personnel's typical product. The recommendations may be individualized at any level, such as medical personnel level, group/department level, health care facility level, or generalized to all parties.

The recommendations provided by an analysis may include recommendations with respect to adjusting an existing product. Adjustments to a product may include any type of adjustment, such as adjustment to dimensions, proportions, shape, materials, instructions for usage, components, or any other type of adjustment. For example, for a particular product, the analysis system may notice that medical personnel hold a medical product at an awkward angle while using it, and it may be desirable to change an angle to a component of the medical product to allow for a more natural ergonomic hold of the product. In another example, for a particular product, the analysis system may show that of the sizes available (e.g., Size 4 and Size 5), medical personnel may seem to require a size that is in between, and may recommend a resized product that may fall between existing sizes (e.g., Size 4.5), which may fit a significant population of the medical personnel.

Such recommendations may be provided with any degree of specificity. For example, they may be provided as high level recommendations. For example, high level recommendations, as ‘make component X larger’ or ‘use a material with higher tensile strength’ or any other type of recommendation may be provided. In some instances, the recommendations may be provided with higher degrees of specificity. In another example, the analysis system may generate an image of the adjustment to the product. The image may be a two-dimensional and/or three-dimensional image of the adjustment to the product. A three-dimensional image may be rotated or viewed from multiple angles. In some instances, the image for the adjustment to the product may be overlaid or presented in a side-by-side manner with an original image of the product. This may allow a user to visualize the adjustment.

The recommendations provided by an analysis may include recommendations with respect to creating an entirely new product. Creation of a new product may include formulation of a product with certain dimensions, proportions, shape, materials, instructions for usage, components, or any other type of specification. The new product may be created to perform a particular functionality. Functionally equivalent products may or may not exist. In some instances, a need for a particular product may be identified based on the analyzed data. For example, during a medical procedure, it may be noted that medical personnel are having difficulty with a particular step or spending a long time on a particular step. A product may be automatically designed that may aid in performing the step. In some instances, a need may be identified based on a large dataset. In some instances, the need may need to surpass a threshold or margin in order to warrant a design of a new product.

Such recommendations for a new product (e.g., new tool creation) may be provided with any degree of specificity. For example, they may be provided as high level recommendations. For example, high level recommendations, as ‘product that can perform Step A including at least components X, Y, and Z’ or any other type of recommendation may be provided. In some instances, the recommendations may be provided with higher degrees of specificity. In another example, the analysis system may generate an image of the new product. The image may be a two-dimensional and/or three-dimensional image of the adjustment to the product. A three-dimensional image may be rotated or viewed from multiple angles. In some instances, the image for the new product may be overlaid or presented in a side-by-side manner with functionally equivalent products. If no functionally equivalent products exist, the image for the new product may be presented with an existing product that is closest to the new product.

When recommending a new product or making adjustments to an existing product, one or more factors may be considered. For example, the factors may include functionality of the product, manufacturing ease of the product, cost of materials of the product, sustainability of the product, predictions relating to usage of the product, predictions pertaining to profits and/or cost of the product, marketability of the product, or any other factors.

In some embodiments, recommendations for adjustments to an existing product or creation of a new product may be made when a sufficient need is identified. In some instances, for sufficient need, when a sufficient percentage of the population would benefit from the product, or a sufficient degree of desired outcome will be realized, then the recommendation may be made. In some instances, one or more thresholds may be set to determine whether a sufficient need exists. The threshold may relate to the number of patients with improved outcomes, the degree of improved outcomes to patients, the number of medical personnel that would utilize the product, the profits to the manufacturers in order to create such a product, or any other factors or combinations thereof.

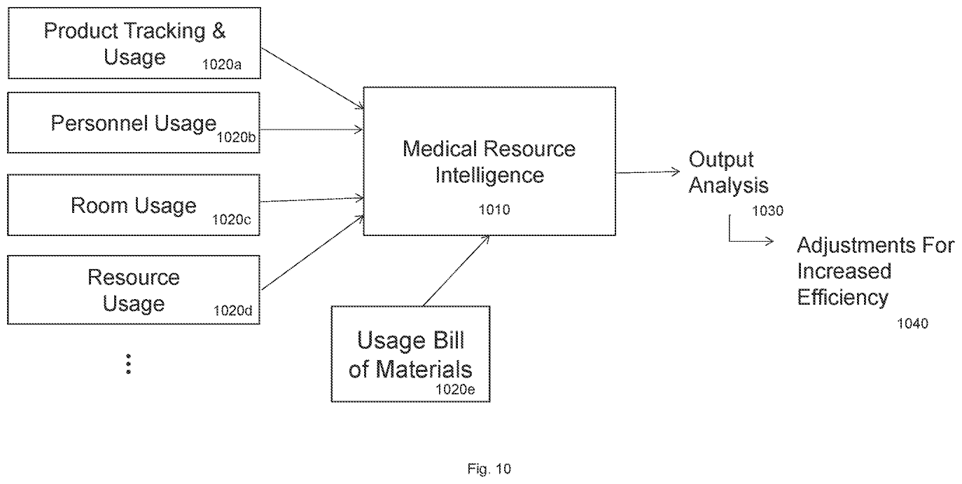

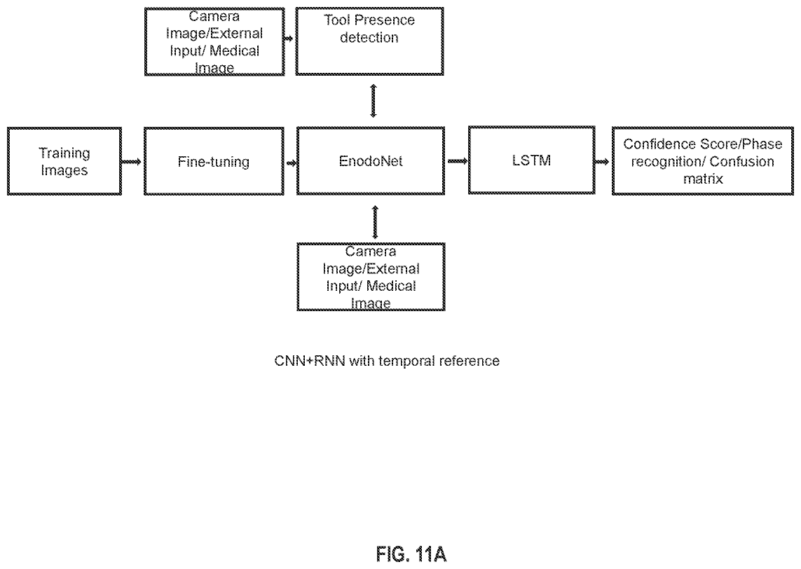

FIG. 10 shows an example of recommendations that may be provided by a medical resource intelligence system for improved performance of a procedure, in accordance with embodiments of the invention.

A medical resource intelligence system 1010 may receive one or more inputs. A medical resource intelligence system may be part of an analysis system or may communicate with an analysis system. In some instances, the medical resource intelligence system may be the analysis system. The one or more inputs may include information relating to procedures or overall usage at a health care facility. Examples of such inputs may include, but are not limited to, product tracking and usage 1020a, personnel usage 1020b, room usage 1020c, resource usage 1020d, or any other type of usage.

Product tracking and usage may include information about the products that are used for various medical procedures. This may include information about particular product types, or the specific brand/model of the product used. In some instances, each product may be individually trackable and information about each individual product used may be tracked (e.g., each product may have a unique serial number, etc.).

Personnel usage may include information about identities of medical personnel that may be performing a procedure. For example, identities of surgeons, physicians' assistants, surgical assistants, nurses, and so forth may be tracked. Information relating to the number of procedures, the length of time of the procedures, and/or outcomes from the procedures may be tracked.

Personnel usage may optionally include information about identities of remote users that may provide support prior to, during, or after a procedure. For example, identities of vendor representatives, specialists, technicians, or any other type of individual that may provide support may be tracked. Information relating to the number of procedures, the length of support for the procedures, length of the procedures, and/or outcomes from the procedures may be tracked. Personnel usage may relate to any human resource that may be utilized.

Room usage may include information about locations where procedures may occur. For example, the various procedures that occur at a particular location may be tracked. The type of procedure, specific identity of the procedure, length of time that the room was used, specifications of the room, and so forth may be tracked.

Resource usage may include any type of resource that may be utilized during a procedure. This may include utilities (e.g., electricity, water, gas, etc.), or other type of resources (e.g., data, connectivity, bandwidth, etc.).

In some instances, data collected prior to, during, or after a procedure may be used to aid in tracking resource usage. For example, video capture systems may capture images of products, personnel, remote users, location, or any other type of resource. The system may automatically identify or track the resources used. The system may track how or when the resources are used, or whether they are used at all.

The system may track and/or count the presence and/or use of resources. For example, the system may track and count the presence or use of medical products. Video data may be used to track and/or count the presence or use of medical products. The use of video data to identify and track the products may advantageously not require adjustments to the products or extra steps. For instance, it does not require scanning of a product when the product is used, does not require manual entry of data, or extra tags (e.g., RFID) on the products or packaging itself. In one example, the system may be able to identify ultimately how the medical product is used by the end of the procedure (e.g., disposed after use, still within the patient, never used at all, etc.). This may be useful for making sure that no unwanted products remain within the patient.

In another example, facial recognition, audio recognition, biometric recognition, or other types of recognition may be used to identify the individuals involved in the procedure, locally or remotely. This may advantageously allow for the identities to be automatically confirmed without requiring further steps by the personnel. The presence or actions of the medical personnel may be analyzed. This may ensure that the medical personnel is present and performing the actions that he or she should be practicing. The amount of time that the medical personnel is present and/or performing steps of the procedure may be tracked and/or analyzed. This may help keep track of shift counts, and be useful to aid in billing or insurance purposes.

A usage bill of materials 1020e may optionally be included. For example, a usage bill relating to any product or resource that may be used may be provided. A usage bill may include information relating to costs relating to any type of product or resource that may be utilized. Everything that may be accountable or non-accountable may be logged, monitored, and/or analyzed by the medical resource intelligence system.

The system may output an analysis 1030. The output may include information relating to the product/resource usage and/or associated costs. In some instances, one or more recommendations may be made.

The recommendations provided by a medical resource intelligence system may include recommendations with respect to adjustments that may be made with respect to resources for a procedure or procedure type. Adjustments to resource usage may include any type of adjustment, such as adjustment to products used, medical personnel participating, remote users participating, location, or any other type of adjustment. The medical resource intelligence system may make recommendations to yield the desired results. Desired results may be based on one or more factors, such as increased efficiency, lower cost, quicker procedure time, patient outcomes, or any factors or combinations thereof

Such recommendations may be provided with any degree of specificity. For example, they may be provided as high level recommendations. For example, high level recommendations, as ‘have Procedure Type A performed in Room 15’ or ‘have Dr. X perform Procedure Type B’ or any other type of recommendation may be provided. In some instances, the recommendations may be provided with higher degrees of specificity. In another example, the system may generate details about the steps to be performed for a procedure and the exact products that should be used for the procedure.

Such analysis may occur prior to a procedure, during a procedure, or after a procedure. After a procedure has been completed, the feedback may be provided to allow for improved procedures in the future. Details of how a past procedure may have been performed differently may be provided. For example, the systems and methods provided herein may suggest an adjustment to a resource that was used or how the resource is to be used for a past procedure. This may advantageously allow for improved efficiency and other desired results in the future.

As described elsewhere herein, the medical resource intelligence system may be configured to track and monitor tool usage information and inventory information. In some cases, the medical resource intelligence system may be configured to generate one or more recommendations for a current procedure or a future procedure based on the tool usage information and/or the inventory information. For example, the medical resource intelligence system may be configured to generate one or more recommendations for which tools or instruments to use for a current or future procedure, or what types or variations of medical techniques to use for a procedure, based on the tool usage information and/or the inventory information.

In some cases, the one or more recommendations may be generated based on one or more annotations or telestrations provided on an image or a video of a surgical procedure. In some instances, a first user (e.g., a first doctor or surgeon or medical specialist) can provide and share telestrations to show how a procedure should be completed. In some cases, a second user (e.g., a second doctor or surgeon or medical specialist) can provide separate telestrations (e.g., telestrations provided on a separate recording or a separate stream/broadcasting channel) to allow a third user (e.g., a third doctor or surgeon or medical specialist) to compare and contrast the various telestrations. In other cases, a second user (e.g., a second doctor or surgeon or medical specialist) can provide telestrations on top of the first user's telestrations to allow a third user (e.g., a third doctor or surgeon or medical specialist) to compare and contrast the various telestrations for a particular video recording, video stream, or video broadcast of a procedure. In some cases, the medical resource intelligence system may be configured to generate one or more recommendations for which tools or instruments to use for a current or future procedure, or what types or variations of medical techniques to use for a procedure, based on the telestrations provided by one or more users viewing an image or a video of a procedure.

In some cases, the medical resource intelligence system may be configured to generate one or more recommendations for which tools or instruments to use for a current or future procedure, or what types or variations of medical techniques to use for a procedure, based on multiple sets of telestrations provided by one or more users viewing an image or a video of a procedure. Such multiple sets of telestrations may be simultaneously generated, streamed to, and/or viewable by various users to compare and contrast various methods and guidance suggested or outlined by the various telestrations provided by the multiple users. In some cases, such multiple sets of telestrations may be simultaneously streamed to and viewable by various users to evaluate different ways to perform one or more steps of the surgical procedure to obtain different results (e.g., different surgical outcomes, or differences in operator efficiency or risk mitigation). In some cases, such multiple sets of telestrations may be simultaneously streamed to and viewable by various users so that the various users can see one or more improvements that can result from performing the surgical procedure in different ways according to the different telestrations provided by different users.

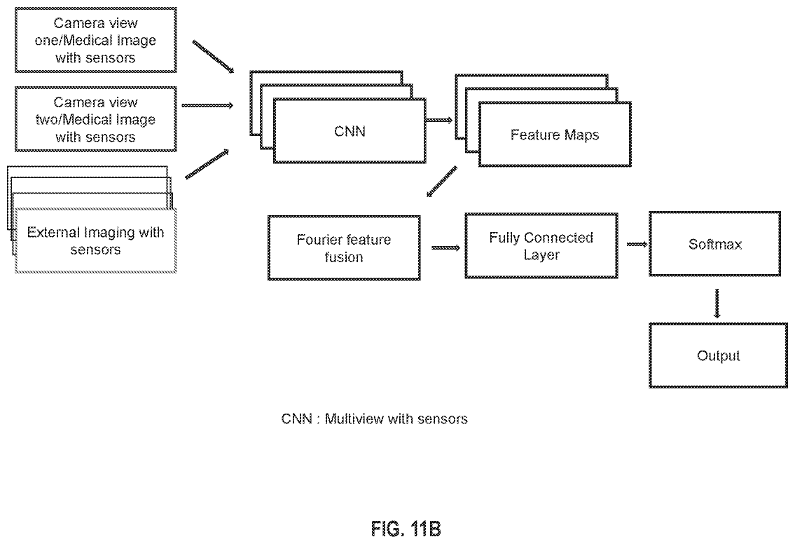

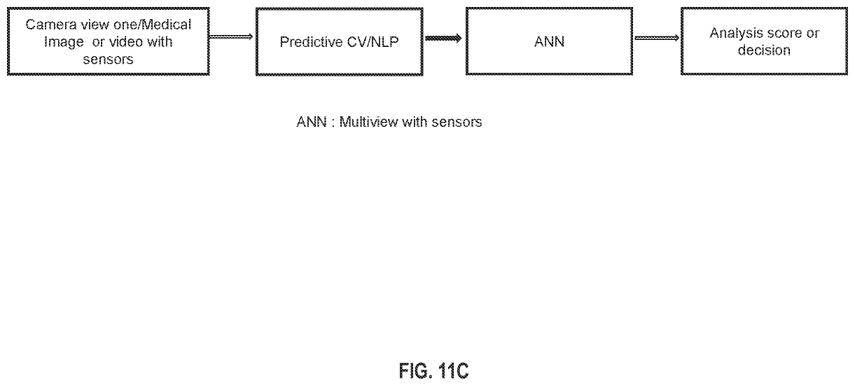

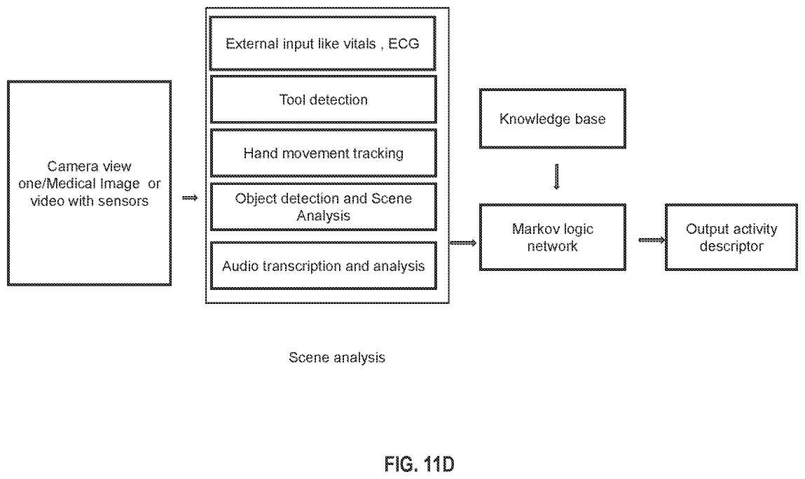

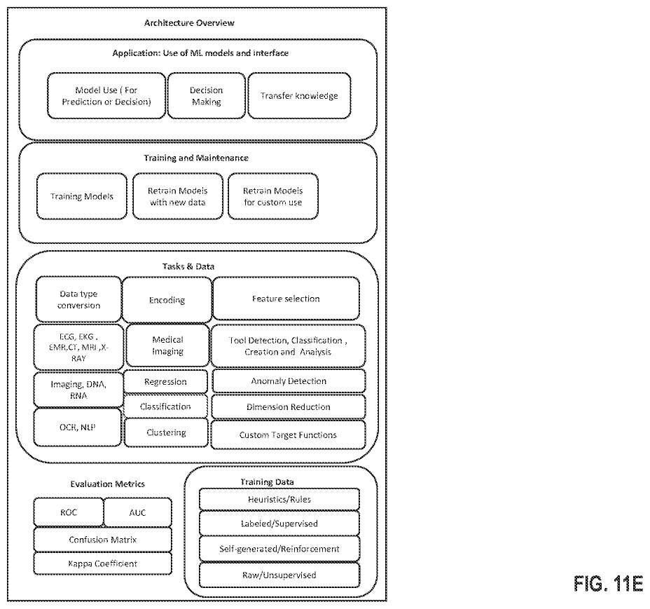

FIGS. 11A-D show examples of various machine learning techniques that may be utilized, in accordance with embodiments of the invention. Machine learning may be utilized by any of the systems and for any of the steps provided herein. For instance, machine learning may be used for video and/or audio recognition. For example, machine learning may be utilized to recognize medical resources, conditions, or steps. Machine learning may be used for analysis and providing recommendations, such as step determination and recognition, in accordance with embodiments of the invention. Any description herein of machine learning may apply to artificial intelligence, and vice versa, or any combination thereof. One or more data sets may be provided. Machine learning data may be generated based on the data sets. The learning data may be useful for recognition, step prediction, and timing prediction. Machine learning may be useful for step recognition and timing recognition as well. The data from such applications may be fed back into the data sets to improve the machine learning algorithms.

One or more data sets may be provided. In some embodiments, data sets may advantageously include a large number of examples collected from multiple sources. In some embodiments, the video analysis system may be in communication with multiple health care facilities and may collect data over time regarding procedures. The data sets may include anatomical data about the patients, medical resources, procedures performed and associated timing information with the various steps of the procedures. As medical personnel perform additional procedures, data relating to these procedures (e.g., anatomy information, procedure/step information, and/or timing information) may be constantly updated and added to the data sets. This may improve the machine learning algorithm and subsequent predictions over time.

The one or more data sets may be used as training data sets for the machine learning algorithms. Learning data may be generated based on the data sets. In some embodiments, supervised learning algorithms may be used. Optionally, unsupervised learning techniques and/or semi-supervised learning techniques may be utilized in order to generate learning data.

In some embodiments, the machine learning may be used to improve medical resource (e.g., medical products, medical personnel, etc.) recognition and/or patient condition recognition. In some embodiments, video captured from one or more cameras during the medical procedure may be analyzed to detect a medical resource or a condition for a patient. Optionally, audio data, medical records, or inputs by medical personnel may be used in addition or alternatively in order to determine a medical resource or a condition for a patient. In some embodiments, object recognition and/or sizing/scaling techniques may be used to determine a medical resource or a condition a patient. A medical personnel may or may not provide feedback in real-time whether the recognition or predictions using the video analysis was correct. In some embodiments, the feedback may be useful for improving recognition in the future.

In some embodiments, the various steps for a medical procedure may be recommended/predicted using a machine learning algorithm. In some embodiments, video information, audio data, medical records, and/or inputs by medical personnel may be used alone or in combination to predict the steps for the medical procedure to be performed by the medical personnel. In some embodiments, the steps may vary depending on a condition of the patient. Machine learning may be useful for generating a series of recommended/predicted steps for the procedure based on the collected information. Optionally, medical personnel may or may not provide feedback in real-time whether the predicted steps are correct for the particular patient. In some embodiments, the feedback may be useful for improving step prediction in the future. Predictions or recommendations for medical steps may also include predictions or recommendations for medical resources, such as medical products, to be used for the steps.

In some embodiments, the timing of the various steps for a medical procedure may be predicted using a machine learning algorithm. In some embodiments, video information, audio data, medical records, and/or inputs by medical personnel may be used alone or in combination to predict the timing of the steps for the medical procedure to be performed by the medical personnel. In some embodiments, the timing of the steps may vary depending on a condition of the patient. Machine learning may be useful for predicting the timing for each of a series of recommended or predicted steps for the procedure based on the collected information. Optionally, medical personnel may or may not provide feedback in real-time whether the predicted timing of the steps are correct for the particular patient. In some embodiments, the feedback may be useful for improving step timing prediction in the future.

As medical personnel are performing a medical procedure, the various steps for a medical procedure may be recognized using a machine learning algorithm. Recognition of the steps may include recognition of the medical products used during the steps. In some embodiments, video information, audio data, medical records, and/or inputs by medical personnel may be used alone or in combination to recognize the steps for the medical procedure that are being performed by the medical personnel. Machine learning may be useful for detecting and recognizing a series of steps for the procedure based on the collected information. Optionally, medical personnel may or may not provide feedback in real-time whether the detected steps are correct for the particular patient. In some embodiments, the feedback may be useful for improving step recognition in the future.

Similarly, during a medical procedure, the timing for the various steps for a medical procedure may be recognized using a machine learning algorithm. In some embodiments, video information, audio data, medical records, and/or inputs by medical personnel may be used alone or in combination to recognize the timing of the steps for the medical procedure that are being performed by the medical personnel. For instance, the systems and methods provided herein may recognize the time at which various steps are started. The systems and methods provided herein may recognize a length of time it takes for the steps to be completed. The systems and methods provided herein may recognize when the next steps are taken. Machine learning may be useful for detecting and recognizing timing for a series of steps for the procedure based on the collected information. Optionally, medical personnel may or may not provide feedback in real-time whether the timing of the detected steps are correct for the particular patient. In some embodiments, the feedback may be useful for improving step timing recognition in the future.

Machine learning may be useful for additional steps, such as recognizing individuals at the location (e.g., medical personnel) and items (e.g., medical products, medical devices) being used. The systems and methods provided may be able to analyze and identify individuals in the room based on the video frames and/or audio captured. For example, facial recognition, motion recognition, gait recognition, voice recognition may be used to recognize individuals in the room. The machine learning may also be utilized to recognize actions taken by the individuals (e.g., picking up an instrument, medical procedure steps, movement within the location). The machine learning may be utilized to recognize a location of the individual.

In some embodiments, the machine learning may utilize deep convolution neural networks/Faster R-CNN Nast NasNet (COCO). The machine learning may utilize any type of convolutional neural network (CNN) and/or recurrent neural network (RNN). Shift invariant or space invariant neural networks (SIANN) may also be utilized. Image classification, object detection and object localization may be utilized. Any machine learning technique known or later developed in the art may be used. For instance, different types of neural networks may be used, such as Artificial Neural Net(ANN), Convolution Neural Net (CNN), Recurrent Neural Net (RNN), and/or their variants.

The machine learning utilized may optionally be a combination of CNN and RNN with temporal reference, as illustrated in FIG. 11A. Input, such as cameras images, external inputs, and/or medical inputs may be provided to a tool presence detection module. The tool presence detection module may communicate with EnodoNet. Training images may be provided for fine-tuning, which may provide data to EnodoNet. Additional input, such as camera images, external inputs, and medical images may be provided to EnodoNet. The output from EnodoNet may be provided to long short-term memory (LSTM). This may provide an output of a confidence score, phase/step recognition, and/or confusion matrix.

The machine learning may optionally utilize CNN for Multiview with sensors as illustrated in FIG. 11B. In some embodiments, inputs, such as various camera views/medical images with sensors, and/or external imaging with sensors may be provided to a CNN learning module. This may provide output to feature maps, which may in turn undergo Fourier feature fusion. The data may then be conveyed to a fully connected layer, and then be provided to Softmax, and then be conveyed as an output.

In some embodiments, the machine learning as described and applied herein may be an artificial neural network (ANN) as illustrated in FIG. 11C. The Multiview with sensors may be provided as illustrated. For instance, an input, such as one or more camera views/medical image or video with sensors may be provided to a predictive (computer vision/natural language processing) CV/NLP module. The output may be conveyed to an ANN module. The output from the ANN may be an analysis score or decision.

FIG. 11D shows an example of scene analysis utilizing machine learning, in accordance with embodiments of the invention. An input may comprise one or more camera views and/or medical image or video with sensors. The input may be provided to a module that may perform one or more functions, such as external input like vitals (e.g., ECG), tool detection, hand movement tracking, object detection and scene analysis, and/or audio transcription and analysis. The output from the module may be provided to a Markov logic network. Data from a knowledge base may also be provided to a Markov logic network. The output from the Markov logic network may be an output activity descriptor.

A location for a medical procedure, such as an operating room, may have one or more cameras which can recognize actors and instruments that are being used using deep convolution neural networks/Faster R-CNN Nast NasNet(COCO) where image classification, object detection, and/or object localization may occur. An audio enhancement module, such as a microphone array as described elsewhere herein, may also be provided at the location for the medical procedure, which can capture everything that is spoken and can convert text to speech for documentation. Using beamforming techniques, the systems ad methods provided can identify an individual that is speaking and the content of the speech. In situations where there is no speech, the systems and methods may rely on video/image data to generate documentation. In addition to storing data related to the entire medical procedure and documenting the procedure, the systems ad methods may be able to generate highlights for the documents and surgery which is composed of video and images.

Medical consoles may be installed on-site (e.g., surgery rooms) which may have multiple cameras and video/audio feeds along with all the skills and tools required to conduct a medical procedure. A separate video feed may be generated in real-time where the next steps that a medical practitioner should be doing along with analysis of the surgery which is going on. This may function as a surgery navigator for doctors. These instructions and video feed that is generated may be played slowly or quickly by adjusting context and scenario of the surgery room. The systems and methods may continuously learn new procedures, surgeries and continuously add data sets which can be used in following medical procedures. These data sets and intelligence may be shared across multiple medical consoles in real-time either through the cloud, P2P or P2P multicast. In addition, the systems and methods provided may be able to add context intelligence and data sets through the platform which can be used by these consoles in real-time.

FIG. 11E shows an example of an architecture of the system, in accordance with some embodiment of the present disclosure. The system may include an application module implementing one or more trained predictive models, a training and maintenance module for training and managing the one or more predictive models, and a tasks and data module for managing various data utilized by the system and one or more databases for storing data related to the one or more predictive models.

The training and maintenance module may be configured for training, developing, deploying and managing the predictive or detective models. In some cases, the training and maintenance module may comprise a model creator and a model manager. In some cases, a model creator may be configured to train, develop or test a predictive or detective model using data from a cloud data lake and/or metadata database that stores contextual data (e.g., deployment context). The model manager may be configured to manage data flows among the various components (e.g., cloud data lake, metadata database, local database, model creator), provide precise, complex and fast queries (e.g., model query, metadata query), model deployment, maintenance, monitoring, model update, model versioning, model sharing, and various others.

The training and maintenance module may be configured to train and develop predictive models. In some cases, the trained predictive models may be deployed to the application module through a predictive model update module. The predictive model update module may monitor the performance of the trained predictive models after deployment and may retrain a model if the performance drops below a pre-determined threshold. In some cases, the training and maintenance module may also support ingesting data transmitted from user device or other data sources into one or more databases or cloud storages for continual training of one or more predictive models

In some cases, the training and maintenance module may include applications that allow for integrated administration and management, including monitoring or storing of data in the cloud or at a private data center. In some embodiments, the training and maintenance module may comprise a user interface (UI) module for monitoring predictive model performance, and/or configuring a predictive model. For instance, the UI module may render a graphical user interface on a computing device allowing a user to view the model performance, or provide user feedback.

The tasks and data management module may be configured to store, search, retrieve, and/or analyze data and information stored in one or more databases. The data and information may include, for example, input data such as ECG, EKG, EMR, CT, MRI, Z-ray data, medical imaging, algorithms or trained models such as OCR, NLP, encoding, regression, classification, clustering, feature selection, tool detection, classification, creation and analysis, anomaly detection, dimension reduction, data about a predictive model (e.g., parameters, model architecture, training dataset, performance metrics, threshold, etc.), data generated by a predictive model, or custom target functions. The data base may store custom models & datasets, standard models and dataset like. The database may store various types of models such as GoogLeNet, AlaxNet ,CLU-CNN, ImageNet , LeNet-5 , DCNN, COINS, TCIA, DDSM,MIAS,VGG16, ukbiobank , Faster R-CNN, Deep residual learning for image recognition, feature pyramid networks for object detection, DSOD, Top down modulation for object detection.

The one or more databases may also store evaluation metrics or performance metrics for a predictive model, training datasets, threshold, rules, and various other data as described elsewhere herein. The one or more trained models may be implemented by the application module to perform various functions and operations consistent with those described herein.

Computer Control Systems

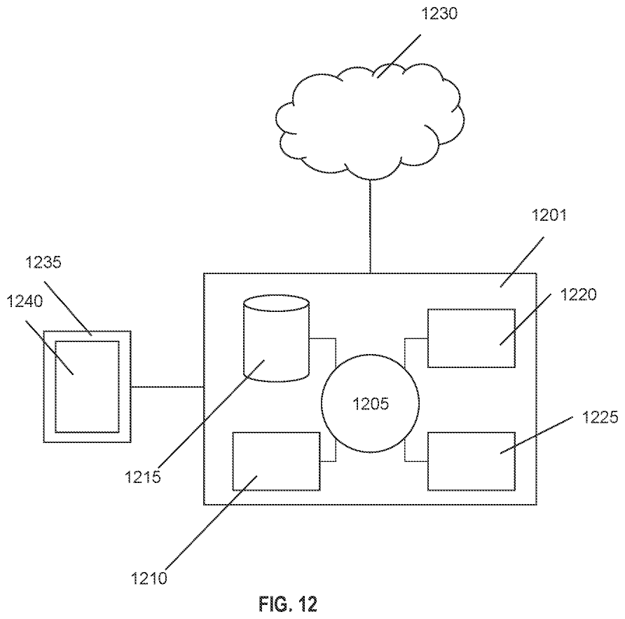

The present disclosure provides computer control systems that are programmed to implement methods of the disclosure. FIG. 12 shows a computer system 1201 that is programmed or otherwise configured to facilitate communications between remote user and medical personnel that may need a remote user's support. The computer system may facilitate communications between a rep communication device and a local communication device. The computer system may automatically interface with one or more medical resource systems of one or more health care facilities. The computer system may analyze data collected at the procedure location, such as video data, audio data, data that may be inputted into a device, and may automatically recognize conditions or steps, and provide recommendations. The computer system can be an electronic device of a user or a computer system that is remotely located with respect to the electronic device. The electronic device can be a mobile electronic device.

The computer system 1201 may include a central processing unit (CPU, also “processor” and “computer processor” herein) 1205, which can be a single core or multi core processor, or a plurality of processors for parallel processing. The computer system also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet and/or extranet, or an intranet and/or extranet that is in communication with the Internet.

The network 1230 in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, capturing a configuration of one or more experimental environments; storing in a registry the experimental environments at each of one or more time points; performing one or more experimental executions which leverage experimental environments; providing outputs of experimental executions which leverage the environments; generating a plurality of linkages between the experimental environments and the experimental executions; and generating one or more execution states corresponding to the experimental environments at one or more time points. Such cloud computing may be provided by cloud computing platforms such as, for example, Amazon Web Services (AWS), Microsoft Azure, Google Cloud Platform, and IBM cloud. The network, in some cases with the aid of the computer system 1201, can implement a peer-to-peer network, which may enable devices coupled to the computer system to behave as a client or a server.

The CPU 1205 can execute a sequence of machine-readable instructions, which can be embodied in a program or software. The instructions may be stored in a memory location, such as the memory 1210. The instructions can be directed to the CPU, which can subsequently program or otherwise configure the CPU to implement methods of the present disclosure. Examples of operations performed by the CPU can include fetch, decode, execute, and writeback.

The CPU 1205 can be part of a circuit, such as an integrated circuit. One or more other components of the system can be included in the circuit. In some cases, the circuit is an application specific integrated circuit (ASIC).

The storage unit 1215 can store files, such as drivers, libraries and saved programs. The storage unit can store user data, e.g., user preferences and user programs. The computer system 1201 in some cases can include one or more additional data storage units that are external to the computer system, such as located on a remote server that is in communication with the computer system through an intranet or the Internet.

The computer system 1201 can communicate with one or more remote computer systems through the network 1230. For instance, the computer system can communicate with a remote computer system of a user (e.g., a user of an experimental environment). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240 for providing, for example, selection of an environment, a component of an environment, or a time point of an environment. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1205. The algorithm can, for example, capture a configuration of one or more experimental environments; store in a registry the experimental environments at each of one or more time points; perform one or more experimental executions which leverage experimental environments; provide outputs of experimental executions which leverage the environments; generate a plurality of linkages between the experimental environments and the experimental executions; and generate one or more execution states corresponding to the experimental environments at one or more time points.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

We claim:

1. A method of forecasting usage of one or more medical resources, said method comprising:
collecting, with aid of one or more video systems, images or videos of a patient during a procedure at a health care location;
analyzing, with aid of one or more processors the images or videos collected with aid of the one or more video systems of the patient during the procedure at the health care location;
recognizing, with aid of the one or more processors, a medical condition of the patient based on the analyzed images or videos collected by the video systems; and
alerting medical personnel to the recognized medical condition.

2. The method of claim 1, wherein the medical condition is previously unknown or undetected for the patient.

3. (canceled)

4. The method of claim 1, further comprising generating and recommending, with aid of the one or more processors, next steps for the procedure, based on the images collected or audio data collected during the procedure.

5. The method of claim 1, further comprising detecting and identifying, with aid of the one or more processors, one or more medical products during the procedure based on the images collected or audio data collected during the procedure.

6. The method of claim 5, wherein the one or more medical products comprises one or more medical tools or instruments.

7. The method of claim 5, further comprising recommending, with aid of the one or more processors, one or more medical products to use during the procedure.

8. The method of claim 5, further comprising detecting or tracking, with aid of the one or more processors, a usage or an operation of the one or more medical products during the procedure, based on the images collected or audio data collected during the procedure.

9. The method of claim 5, further comprising recommending one or more optimal ways for performing one or more steps of the procedure based on the detection or identification of the one or more medical products.

10. (canceled)

11. (canceled)

12. (canceled)

13. The method of claim 1, further comprising generating or updating one or more recommendations for the procedure based on a change in the recognized condition.

14. The method of claim 13, wherein the one or more recommendations comprise a recommendation for a specific product, a particular medical operator, or a certain medical technique.

15. (canceled)

16. The method of claim 1, further comprising generating one or more recommendations for the procedure based on data from auxiliary sources, wherein the auxiliary sources comprise endoscopes, laparoscopes, electrocardiogram (ECG) devices, heartbeat monitors, or pulse oximeters.

17. (canceled)

18. The method of claim 1, further comprising generating one or more recommendations for future procedures based on an analysis of a past procedure, wherein the one or more recommendations comprise a variation of a medical technique performed in the past procedure.

19. (canceled)

20. The method of claim 1, further comprising predicting an outcome for the procedure based on the recognized condition and one or more input parameters, wherein the one or more input parameters comprise a medical condition of the patient, one or more tools used to perform the procedure, an identity of medical personnel performing or assisting with the procedure, an identity of remote users, a location of the procedure, or one or more techniques used to perform one or more steps of the procedure.

21. (canceled)

22. The method of claim 1, further comprising recommending one or more products based on a comparison between outcomes or results associated with a plurality of different products.

23. A method of formulating product recommendations, said method comprising:
collecting, with aid of one or more video or audio systems, images, video, or audio of a patient during a procedure at a health care location;
analyzing, with aid of one or more processors the images, video, or audio collected with aid of the one or more video or audio systems of the patient during the procedure at the health care location; and
recommending, with aid of one or more processors, one or more new medical products or modifications to one or more existing medical products based on the analysis of the images, video, or audio collected during the procedure.

24. (canceled)

25. The method of claim 23, further comprising recommending one or more functionally equivalent products associated with the one or more existing medical products.

26. The method of claim 23, wherein the recommendations for the one or more new medical products or the suggestions for modifying the one or more existing medical products are generated based on an analysis of patient outcomes associated with the new or existing medical products.

27. The method of claim 23, wherein the recommendations for the one or more new medical products or the suggestions for modifying the one or more existing medical products are generated based on one or more factors associated with product functionality, product usage rate, or cost.

28. (canceled)

29. The method of claim 23, further comprising updating the recommendations in real time based on an analysis of additional images, video, or audio collected during the procedure.

30. (canceled)

31. The method of claim 23, further comprising using a machine learning algorithm to generate the recommendations for the one or more new medical products or the modifications to the one or more existing medical products.
